SEQUENCE LISTING (98 sequences)

SEQ ID NO: 1 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcattaatataagatgtaatgacaagtgctactgtgaagatggctatgcaagggatgtcaatggcaaatgtataccgataaaagactgtcctaaaatacgttcg

SEQ ID NO: 2 - protein, 69 aa, Galleria mellonella
IVLICNGGHEYYECGGACDNVCADLHIQNKTNCPIINIRCNDKCYCEDGYARDVNGKCIPIKDCPKIRS

SEQ ID NO: 3 - DNA, 249 bp, Galleria mellonella
  CDS (1)..(249)
cgtcgttccattgggataccagtcgacaagaaatgctgcacaggtcctaacgaacactatgacgaagagaaagtaagctgtcctccagaaacctgtatctcccttgtggctaagttttcctgcattgactcccctccaccgtcgccagggtgttcttgcaattcaggatacttaagacttaacctaacttcaccatgcataccaatttgcgattgtccacaaatgcaacattcccctgattgtcaataa

SEQ ID NO: 4 - protein, 82 aa, Galleria mellonella
RRSIGIPVDKKCCTGPNEHYDEEKVSCPPETCISLVAKFSCIDSPPPSPGCSCNSGYLRLNLTSPCIPICDCPQMQHSPDCQ

SEQ ID NO: 5 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtcccatcattaatataagatgtaatgacaagtgctactgtgaagatggctatgcaagggatgtcaatggcaaatgtataccgataaaagactgtcctaaaatacgttcgtag

SEQ ID NO: 6 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPIINIRCNDKCYCEDGYARDVNGKCIPIKDCPKIRS

SEQ ID NO: 7 - DNA, 1080 bp, Galleria mellonella
  CDS (1)..(1080)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagcgtcgttccattgggataccagtcgacaagaaatgctgcacaggtcctaacgaacactatgacgaagagaaagtaagctgtcctccagaaacctgtatctcccttgtggctaagttttcctgcattgactcccctccaccgtcgccagggtgttcttgcaattcaggatacttaagacttaacctaacttcaccatgcataccaatttgcgattgtccacaaatgcaacattcccctgattgtcaatag

SEQ ID NO: 8 - protein, 359 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKRRSIGIPVDKKCCTGPNEHYDEEKVSCPPETCISLVAKFSCIDSPPPSPGCSCNSGYLRLNLTSPCIPICDCPQMQHSPDCQ

SEQ ID NO: 9 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcattggtctggcttgtaatgacaagtgctactgtgaagatggctatgcaagggatgtcaatggcaaatgtataccgataaaagactgtcctaaaatacgttcg

SEQ ID NO: 10 - protein, 69 aa, Galleria mellonella
IVLICNGGHEYYECGGACDNVCADLHIQNKTNCPIIGLACNDKCYCEDGYARDVNGKCIPIKDCPKIRS

SEQ ID NO: 11 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtcccatcattggtctggcttgtaatgacaagtgctactgtgaagatggctatgcaagggatgtcaatggcaaatgtataccgataaaagactgtcctaaaatacgttcgtag

SEQ ID NO: 12 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPIIGLACNDKCYCEDGYARDVNGKCIPIKDCPKIRS

SEQ ID NO: 13 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcattaatataagatgtaatgacaagtgctactgtgaagatggctatgcaaaggatgtcaatggcaaatgtataccgataaaagactgtcctaaaatacgttcg

SEQ ID NO: 14 - protein, 69 aa, Galleria mellonella
IVLICNGGHEYYECGGACDNVCADLHIQNKTNCPIINIRCNDKCYCEDGYAKDVNGKCIPIKDCPKIRS

SEQ ID NO: 15 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtcccatcattaatataagatgtaatgacaagtgctactgtgaagatggctatgcaaaggatgtcaatggcaaatgtataccgataaaagactgtcctaaaatacgttcgtag

SEQ ID NO: 16 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPIINIRCNDKCYCEDGYAKDVNGKCIPIKDCPKIRS

SEQ ID NO: 17 - DNA, 207 bp, Galleria mellonella
  CDS (1)..(207)
  misc_feature (185)..(185): n is a, c, g, or t
atagtcctaatttgtaacggtggacacgaatactacgagtgcggtggagcctgcgataatgtatgtgcagatttacatatacagaataaaacaaactgtcccttgattaatataagatgtaatgacaagtgctactgtgaagatggctatgcaagggatgtcaatggcaaatgtataccgataanagactgtcctaaaatacgttcg

SEQ ID NO: 18 - protein, 69 aa, Galleria mellonella
  misc_feature (62)..(62): The ′Xaa′ at location 62 stands for Lys, Arg, Thr, or Ile.
IVLICNGGHEYYECGGACDNVCADLHIQNKTNCPLINIRCNDKCYCEDGYARDVNGKCIPIXDCPKIRS

SEQ ID NO: 19 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatgattaatataagatgtaatgacaagtgctactgtgaagatggctatgcaagggatgtcaatggcaaatgtataccgataaaagactgtcctaaaatacgttcg

SEQ ID NO: 20 - protein, 69 aa, Galleria mellonella
IVLICNGGHEYYECGGACDNVCADLHIQNKTNCPMINIRCNDKCYCEDGYARDVNGKCIPIKDCPKIRS

SEQ ID NO: 21 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccttcattaatataagatgtaatgacaagtgctactgtgaagatggctatgcaagggatgtcaatggcaaatgtataccgataaaagactgtcctaaaatacgttcg

SEQ ID NO: 22 - protein, 69 aa, Galleria mellonella
IVLICNGGHEYYECGGACDNVCADLHIQNKTNCPFINIRCNDKCYCEDGYARDVNGKCIPIKDCPKIRS

SEQ ID NO: 23 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtccctgtattaatataagatgtaatgacaagtgctactgtgaagatggctatgcaagggatgtcaatggcaaatgtataccgataaaagactgtcctaaaatacgttcg

SEQ ID NO: 24 - protein, 69 aa, Galleria mellonella
IVLICNGGHEYYECGGACDNVCADLHIQNKTNCPCINIRCNDKCYCEDGYARDVNGKCIPIKDCPKIRS

SEQ ID NO: 25 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccaatattaatataagatgtaatgacaagtgctactgtgaagatggctatgcaagggatgtcaatggcaaatgtataccgataaaagactgtcctaaaatacgttcg

SEQ ID NO: 26 - protein, 69 aa, Galleria mellonella
IVLICNGGHEYYECGGACDNVCADLHIQNKTNCPNINIRCNDKCYCEDGYARDVNGKCIPIKDCPKIRS

SEQ ID NO: 27 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtccccagattaatataagatgtaatgacaagtgctactgtgaagatggctatgcaagggatgtcaatggcaaatgtataccgataaaagactgtcctaaaatacgttcg

SEQ ID NO: 28 - protein, 69 aa, Galleria mellonella
IVLICNGGHEYYECGGACDNVCADLHIQNKTNCPQINIRCNDKCYCEDGYARDVNGKCIPIKDCPKIRS

SEQ ID NO: 29 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtccccacattaatataagatgtaatgacaagtgctactgtgaagatggctatgcaagggatgtcaatggcaaatgtataccgataaaagactgtcctaaaatacgttcg

SEQ ID NO: 30 - protein, 69 aa, Galleria mellonella
IVLICNGGHEYYECGGACDNVCADLHIQNKTNCPHINIRCNDKCYCEDGYARDVNGKCIPIKDCPKIRS

SEQ ID NO: 31 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccaagattaatataagatgtaatgacaagtgctactgtgaagatggctatgcaagggatgtcaatggcaaatgtataccgataaaagactgtcctaaaatacgttcg

SEQ ID NO: 32 - protein, 69 aa, Galleria mellonella
IVLICNGGHEYYECGGACDNVCADLHIQNKTNCPKINIRCNDKCYCEDGYARDVNGKCIPIKDCPKIRS

SEQ ID NO: 33 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtccccgtattaatataagatgtaatgacaagtgctactgtgaagatggctatgcaagggatgtcaatggcaaatgtataccgataaaagactgtcctaaaatacgttcg

SEQ ID NO: 34 - protein, 69 aa, Galleria mellonella
IVLICNGGHEYYECGGACDNVCADLHIQNKTNCPRINIRCNDKCYCEDGYARDVNGKCIPIKDCPKIRS

SEQ ID NO: 35 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcgttaatataagatgtaatgacaagtgctactgtgaagatggctatgcaagggatgtcaatggcaaatgtataccgataaaagactgtcctaaaatacgttcg

SEQ ID NO: 36 - protein, 69 aa, Galleria mellonella
IVLICNGGHEYYECGGACDNVCADLHIQNKTNCPIVNIRCNDKCYCEDGYARDVNGKCIPIKDCPKIRS

SEQ ID NO: 37 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcatgaatataagatgtaatgacaagtgctactgtgaagatggctatgcaagggatgtcaatggcaaatgtataccgataaaagactgtcctaaaatacgttcg

SEQ ID NO: 38 - protein, 69 aa, Galleria mellonella
IVLICNGGHEYYECGGACDNVCADLHIQNKTNCPIMNIRCNDKCYCEDGYARDVNGKCIPIKDCPKIRS

SEQ ID NO: 39 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcttcaatataagatgtaatgacaagtgctactgtgaagatggctatgcaagggatgtcaatggcaaatgtataccgataaaagactgtcctaaaatacgttcg

SEQ ID NO: 40 - protein, 69 aa, Galleria mellonella
IVLICNGGHEYYECGGACDNVCADLHIQNKTNCPIFNIRCNDKCYCEDGYARDVNGKCIPIKDCPKIRS

SEQ ID NO: 41 - DNA, 207 bp, Galleria mellonella
  CDS (1)..(207)
  misc_feature (7)..(7): n is a, c, g, or t
atagtcntaatttgtancggtggacacgaatantnngngtgcggtggagcctgcgataatgtatgtgcagatttacatatacagaannaaacaaactgtcccatctggaatataagatgtaatgacaagtgctactgtgaagatggctatgcaagggatgtcaatggcaaatgtataccgattaangnntgtcctaaaatacgttcg

SEQ ID NO: 42 - protein, 69 aa, Galleria mellonella
  misc_feature (3)..(3): The ′Xaa′ at location 3 stands for Ile, Val, or Leu.
IVXICXGGHEXXXCGGACDNVCADLHIQXXTNCPIWNIRCNDKCYCEDGYARDVNGKCIPIXXCPKIRS

SEQ ID NO: 43 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatctacaatataagatgtaatgacaagtgctactgtgaagatggctatgcaagggatgtcaatggcaaatgtataccgataaaagactgtcctaaaatacgttcg

SEQ ID NO: 44 - protein, 69 aa, Galleria mellonella
IVLICNGGHEYYECGGACDNVCADLHIQNKTNCPIYNIRCNDKCYCEDGYARDVNGKCIPIKDCPKIRS

SEQ ID NO: 45 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcagtaatataagatgtaatgacaagtgctactgtgaagatggctatgcaagggatgtcaatggcaaatgtataccgataaaagactgtcctaaaatacgttcg

SEQ ID NO: 46 - protein, 69 aa, Galleria mellonella
IVLICNGGHEYYECGGACDNVCADLHIQNKTNCPISNIRCNDKCYCEDGYARDVNGKCIPIKDCPKIRS

SEQ ID NO: 47 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcactaatataagatgtaatgacaagtgctactgtgaagatggctatgcaagggatgtcaatggcaaatgtataccgataaaagactgtcctaaaatacgttcg

SEQ ID NO: 48 - protein, 69 aa, Galleria mellonella
IVLICNGGHEYYECGGACDNVCADLHIQNKTNCPITNIRCNDKCYCEDGYARDVNGKCIPIKDCPKIRS

SEQ ID NO: 49 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcaataatataagatgtaatgacaagtgctactgtgaagatggctatgcaagggatgtcaatggcaaatgtataccgataaaagactgtcctaaaatacgttcg

SEQ ID NO: 50 - protein, 69 aa, Galleria mellonella
IVLICNGGHEYYECGGACDNVCADLHIQNKTNCPINNIRCNDKCYCEDGYARDVNGKCIPIKDCPKIRS

SEQ ID NO: 51 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatccagaatataagatgtaatgacaagtgctactgtgaagatggctatgcaagggatgtcaatggcaaatgtataccgataaaagactgtcctaaaatacgttcg

SEQ ID NO: 52 - protein, 69 aa, Galleria mellonella
IVLICNGGHEYYECGGACDNVCADLHIQNKTNCPIQNIRCNDKCYCEDGYARDVNGKCIPIKDCPKIRS

SEQ ID NO: 53 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatccataatataagatgtaatgacaagtgctactgtgaagatggctatgcaagggatgtcaatggcaaatgtataccgataaaagactgtcctaaaatacgttcg

SEQ ID NO: 54 - protein, 69 aa, Galleria mellonella
IVLICNGGHEYYECGGACDNVCADLHIQNKTNCPIHNIRCNDKCYCEDGYARDVNGKCIPIKDCPKIRS

SEQ ID NO: 55 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatccgtaatataagatgtaatgacaagtgctactgtgaagatggctatgcaagggatgtcaatggcaaatgtataccgataaaagactgtcctaaaatacgttcg

SEQ ID NO: 56 - protein, 69 aa, Galleria mellonella
IVLICNGGHEYYECGGACDNVCADLHIQNKTNCPIRNIRCNDKCYCEDGYARDVNGKCIPIKDCPKIRS

SEQ ID NO: 57 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcaagaatataagatgtaatgacaagtgctactgtgaagatggctatgcaagggatgtcaatggcaaatgtataccgataaaagactgtcctaaaatacgttcg

SEQ ID NO: 58 - protein, 69 aa, Galleria mellonella
IVLICNGGHEYYECGGACDNVCADLHIQNKTNCPIKNIRCNDKCYCEDGYARDVNGKCIPIKDCPKIRS

SEQ ID NO: 59 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcattaatatagtatgtaatgacaagtgctactgtgaagatggctatgcaagggatgtcaatggcaaatgtataccgataaaagactgtcctaaaatacgttcg

SEQ ID NO: 60 - protein, 69 aa, Galleria mellonella
IVLICNGGHEYYECGGACDNVCADLHIQNKTNCPIINIVCNDKCYCEDGYARDVNGKCIPIKDCPKIRS

SEQ ID NO: 61 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcattaatataaaatgtaatgacaagtgctactgtgaagatggctatgcaagggatgtcaatggcaaatgtataccgataaaagactgtcctaaaatacgttcg

SEQ ID NO: 62 - protein, 69 aa, Galleria mellonella
IVLICNGGHEYYECGGACDNVCADLHIQNKTNCPIINIKCNDKCYCEDGYARDVNGKCIPIKDCPKIRS

SEQ ID NO: 63 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtccctggattaatataagatgtaatgacaagtgctactgtgaagatggctatgcaagggatgtcaatggcaaatgtataccgataaaagactgtcctaaaatacgttcg

SEQ ID NO: 64 - protein, 69 aa, Galleria mellonella
IVLICNGGHEYYECGGACDNVCADLHIQNKTNCPWINIRCNDKCYCEDGYARDVNGKCIPIKDCPKIRS

SEQ ID NO: 65 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtccctacattaatataagatgtaatgacaagtgctactgtgaagatggctatgcaagggatgtcaatggcaaatgtataccgataaaagactgtcctaaaatacgttcg

SEQ ID NO: 66 - protein, 69 aa, Galleria mellonella
IVLICNGGHEYYECGGACDNVCADLHIQNKTNCPYINIRCNDKCYCEDGYARDVNGKCIPIKDCPKIRS

SEQ ID NO: 67 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccatcattaatatagcatgtaatgacaagtgctactgtgaagatggctatgcaagggatgtcaatggcaaatgtataccgataaaagactgtcctaaaatacgttcg

SEQ ID NO: 68 - protein, 69 aa, Galleria mellonella
IVLICNGGHEYYECGGACDNVCADLHIQNKTNCPIINIACNDKCYCEDGYARDVNGKCIPIKDCPKIRS

SEQ ID NO: 69 - DNA, 207 bp, Galleria mellonella; identical to SEQ ID NO: 23
  CDS (1)..(207)

SEQ ID NO: 70 - protein, 69 aa, Galleria mellonella; identical to SEQ ID NO: 24

SEQ ID NO: 71 - DNA, 207 bp, Galleria mellonella; identical to SEQ ID NO: 31
  CDS (1)..(207)

SEQ ID NO: 72 - protein, 69 aa, Galleria mellonella; identical to SEQ ID NO: 32

SEQ ID NO: 73 - DNA, 207 bp, Galleria mellonella; identical to SEQ ID NO: 33
  CDS (1)..(207)

SEQ ID NO: 74 - protein, 69 aa, Galleria mellonella; identical to SEQ ID NO: 34

SEQ ID NO: 75 - DNA, 207 bp, Galleria mellonella
  CDS (1)..(207)
atagtcctaatttgtaacggtggacacgaatactacgagtgcggtggagcctgcgataatgtatgtgcagatttacatatacagaataaaacaaactgtcccttgattaatataagatgtaatgacaagtgctactgtgaagatggctatgcaagggatgtcaatggcaaatgtataccgataaaagactgtcctaaaatacgttcg

SEQ ID NO: 76 - protein, 69 aa, Galleria mellonella
IVLICNGGHEYYECGGACDNVCADLHIQNKTNCPLINIRCNDKCYCEDGYARDVNGKCIPIKDCPKIRS

SEQ ID NO: 77 - DNA, 207 bp, Galleria mellonella; identical to SEQ ID NO: 19
  CDS (1)..(207)

SEQ ID NO: 78 - protein, 69 aa, Galleria mellonella; identical to SEQ ID NO: 20

SEQ ID NO: 79 - DNA, 207 bp, Galleria mellonella; identical to SEQ ID NO: 21
  CDS (1)..(207)

SEQ ID NO: 80 - protein, 69 aa, Galleria mellonella; identical to SEQ ID NO: 22

SEQ ID NO: 81 - DNA, 207 bp, Galleria mellonella; identical to SEQ ID NO: 27
  CDS (1)..(207)

SEQ ID NO: 82 - protein, 69 aa, Galleria mellonella; identical to SEQ ID NO: 28

SEQ ID NO: 83 - DNA, 207 bp, Galleria mellonella; identical to SEQ ID NO: 29
  CDS (1)..(207)

SEQ ID NO: 84 - protein, 69 aa, Galleria mellonella; identical to SEQ ID NO: 30

SEQ ID NO: 85 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtcccaatattaatataagatgtaatgacaagtgctactgtgaagatggctatgcaagggatgtcaatggcaaatgtataccgataaaagactgtcctaaaatacgttcgtag

SEQ ID NO: 86 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPNINIRCNDKCYCEDGYARDVNGKCIPIKDCPKIRS

SEQ ID NO: 87 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtcccatcagtaatataagatgtaatgacaagtgctactgtgaagatggctatgcaagggatgtcaatggcaaatgtataccgataaaagactgtcctaaaatacgttcgtag

SEQ ID NO: 88 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPISNIRCNDKCYCEDGYARDVNGKCIPIKDCPKIRS

SEQ ID NO: 89 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtcccatcattaatataaaatgtaatgacaagtgctactgtgaagatggctatgcaagggatgtcaatggcaaatgtataccgataaaagactgtcctaaaatacgttcgtag

SEQ ID NO: 90 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPIINIKCNDKCYCEDGYARDVNGKCIPIKDCPKIRS

SEQ ID NO: 91 - DNA, 1041 bp, Galleria mellonella
  CDS (1)..(1041)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtgatgacgacgacaagatagtcctaatttgtaacggtggacacgaatactacgagtgcggtggagcctgcgataatgtatgtgcagatttacatatacagaataaaacaaactgtccctgtattaatataagatgtaatgacaagtgctactgtgaagatggctatgcaagggatgtcaatggcaaatgtataccgataaaagactgtcctaaaatacgttcgtag

SEQ ID NO: 92 - protein, 346 aa, Galleria mellonella
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGDDDDKIVLICNGGHEYYECGGACDNVCADLHIQNKTNCPCINIRCNDKCYCEDGYARDVNGKCIPIKDCPKIRS

SEQ ID NO: 93 - DNA, 57 bp, Galleria mellonella
  CDS (1)..(57)
atgaagtgcttattatatttatgtctatggtgttattgtgtactagtaagcagtagc

SEQ ID NO: 94 - protein, 19 aa, Galleria mellonella
MKCLLYLCLWCYCVLVSSS

SEQ ID NO: 95 - protein, 55 aa, Solenopsis
  PEPTIDE (1)..(55)
CNRENEEYQCGSACQTTCTNLGQNCSIINIRCNDACYCKPGYARMGGDSSPCIPI

SEQ ID NO: 96 - DNA, 32 bp, insect
ctacgaacgtattttaggacagtcttttatcg

SEQ ID NO: 97 - DNA, 28 bp, insect
gatagtcctaatttgtaacggtggacac

SEQ ID NO: 98 - protein, 20 aa, artificial
  An amide of a substrate published in Turk B.E., et al., “The structural basis for substrate and inhibitor selectivity of the anthrax lethal factor” Nature Structural & Molecular
MLARRKKVYPYPMEPTIAEG

(19) United States
(12) Patent Application Publication
(10) Pub. No.: US 2016/0159885 A1
(43) Pub. Date: Jun. 9, 2016

(54) INSECT METALLOPROTEINASE INHIBITORS

(71) Applicant: FRAUNHOFER-GESELLSCHAFT ZUR FÖRDERUNG DER ANGEWANDTEN FORSCHUNG E.V., Munich (DE)

(72) Inventors: Andreas Vilcinskas, Ferwald (DE); Mark Salzig, Gießen (DE); Rainer Fischer, Aachen (DE)

(21) Appl. No.: 14/906,887

(22) PCT Filed: Jul. 28, 2014
(86) PCT No.: PCT/EP2014/066165

§ 371 Date: Jan. 21, 2016

(30) Foreign Application Priority Data
Jul. 26, 2013 (EP) 13178180.9

(51) Int. Cl. C07K 14/81; C07K 16/00; C12Q 1/37; C07K 14/435

(52) U.S. Cl. CPC C07K 14/8146; C07K 14/43563; C07K 16/00; C12Q 1/37; C07K 2319/30; C07K 2319/40; C07K 2319/00; G01N 2333/96419

(57) ABSTRACT

A polypeptide having at least 70% homology, in particular 80%, 90% or 95% homology to the polypeptide of SEQ ID NO:2 representing the wild-type of the protein insect metalloproteinase inhibitor IMPIα and having at least one mutation at position 35, 36 and/or 39 of the amino acid sequence of IMPIα and the polypeptide having an IC₅₀ value to thermolysine of less than the IC₅₀ value of IMPIα wherein

the nonpolar amino acid isoleucine at position 35 of IMPIα is replaced either by a nonpolar amino acid selected from the group consisting of leucine, methionine and phenylalanine or by polar amino acid selected from the group consisting of cysteine, asparagine, glutamine, histidine, lysine and arginine; and/or

the nonpolar amino acid isoleucine at position 36 of IMPIα is replaced either by a nonpolar amino acid selected from the group consisting of valine, phenylalanine and tryptophan or by polar amino acid selected from the group consisting of tyrosine, serine, threonine, asparagine, glutamine, histidine, lysine and arginine; and/or

the polar amino acid position 39 of IMPIα is replaced either by the nonpolar amino acid valine or by the polar amino acids histidine or lysine.

### CROSS-REFERENCE TO RELATED APPLICATION

This application is a U.S. National Phase of International Application No. PCT/EP2014/066165, filed 28 Jul. 2014, which claims priority to European Application No. 13178180.9, filed 26 Jul. 2013, the entire contents of each are incorporated by reference herein.

### SEQUENCE LISTING

This application contains a Sequence Listing which has been submitted electronically in ASCII format and is incorporated by reference in its entirety. Said ASCII copy is named 395293SequenceListing.txt and is 140 KB in size. Said ASCII copy is a copy of the sequence listing filed with International Application No. PCT/EP2014/064307, filed 28 Jul. 2014.

### FIELD OF THE INVENTION

This invention relates to peptides and proteins inhibiting protease activity. More specifically, the invention relates to peptides exhibiting activity against a spectrum of proteases of microbial or fungal origin, in particular against the metzincin family including thermolysin, anthrax neutral protease 599 (npr599), pseudolysin, and aureolysin. The invention relates further to methods for producing the peptides, to pharmaceutical compositions comprising the peptides or proteins, and to methods of using the peptides or proteins to prevent and/or treat bacterial or fungal infections and their symptoms, in particular to reduce the toxic effects of secreted or membrane bound bacterial proteases such as aureolysin, bacillolysin, pseudolysin, vibriolysin, and anthrax npr599 by inhibiting their respective proteolytic activity.

### BACKGROUND OF THE INVENTION

Antibiotics are the actual standard treatment for bacterial infections. The wide-spread and sometimes inadequate use of antibiotics, however, has led to pathogens developing resistance against one or even multiple antibiotics. Multidrug resistant bacteria pose an increasing and serious threat to infected patients. Therefore the search for new companion compounds dampening the bacterial virulence has become an urgent need during the last years. A rapid effect of compounds against dangerous or life threatening symptoms of bacterial infection is desirable to protect the patient immediately against these symptoms even before antibiotics or alternative compounds can kill the microbes.

For decades it has been known that bacteria release proteases (Duthie et al. 1949). Later the pathogenic role of microbial proteases as virulence factors was recognized experimentally (Snell et al. 1978). Proteolytic enzymes produced by pathogens and parasites are essential for their successful infection of the host. Regardless of their substrate specificity the key function of individual pathogen-derived proteinases is the degradation of host proteins and peptides (Armstrong et al. 2006). Extracellular digestion of host proteins facilitates, for example, penetration across physical barriers of the host, for which the degradation of the extracellular matrix may serve as a prominent example. Furthermore, the degradation of immunity-related defense molecules of the host, including antimicrobial peptides (AMP) (Otto 2008), and the acquisition of nutrients are important functions for the pathogen to survive. However, only those pathogen-associated proteolytic enzymes can serve as virulence factors that cannot or can only partially be inactivated by corresponding, endogenous host proteinase inhibitors. Virulence factors usually complement each other in degrading host peptides and proteins during infection. Consequently, pathogen-derived proteinases without corresponding endogenous inhibitors in the host will be evolutionarily selected because pathogens would otherwise waste their limited resources (Vilcinskas 2010).

Mammals and many invertebrates lack specific inhibitors for proteases of the M4 or Metzincin family of metalloproteinases, of which thermolysin from Bacillus thermoproteolyticus is a salient example. Thermolysin-like metalloproteinases encompass many prominent toxins produced by human pathogens, including aureolysin, bacillolysin, pseudolysin, vibriolysin, and anthrax npr599, which are presumably at the origin of many pathological symptoms associated with severe infections such as septicemia, hemorrhagic tissue bleeding, necrosis and enhancement of vascular permeability (Chung et al. 2006). Severe diseases like gastric and peptic ulcers and gastric carcinoma originate at least partly from the effect of metalloproteinases of the M4 family from pathogens (Schmidtchen et al. 2003, Smith et al. 1994).

For example, the major symptoms of an infection with bacillus anthracis are caused by the release of virulence factors from the bacteria. The well known virulence factor anthrax toxin leads to the destruction of the host macrophages, thereby enabling the bacteria to spread in the host tissue without encountering host defense. The anthrax toxin is a holoenzyme containing three different proteins, each of which is present in multiple copies. They are called protective antigen (PA), edema factor (EF), and lethal factor (LF). To be become active, it is required that at least PA and EF or PA and LF are combined. Just a single protein or just EF and LF without PA are not active in laboratory animals.

More recently it became evident that the combined activity of the PA, EF and LF alone does not cover all observed clinical anthrax symptoms. It was shown that anthrax harbors further genes coding for secreted proteins exhibiting proteolytic activity. Amongst those, gene BA0599 from bacillus anthracis (BA) codes for a protease called neutral protease 599 (npr599), which belongs to the M4 family of proteases and is highly homologous to bacillolysins from other bacillus species (Popov et al. 2005, Chung et al. 2006, Chung et al. 2008).

Currently several treatment options for anthrax infections exist. A vaccine is available which protects with a safety of more than 90%, but usually the degree of vaccination against anthrax in a human population is low and so far it is not expected that this degree will increase. Anthrax can further be treated with antibiotics in case of an acute infection. However, the toxins shed by the bacteria continue to cause irreversible damage to tissue before the number of bacteria is sufficiently reduced by antibiotics. It is even likely that exposure to antibiotics stimulates bacteria to temporarily shed increased amounts of virulence factors, causing accelerated and irreversible damage of patient tissue. Furthermore, manmade synthetic or recombinant versions of the natural bacterial toxins may be inhaled by humans without bacteria being present, in which cases an antibiotic would have no curing effect at all.

Therefore the task of inhibiting or antagonizing bacterial toxins directly was recognized as a challenge, and inhibition strategies were published. In the case of Pseudomonas aeruginosa virulence factor LasB (Cathgart G. R. et al, 2011), N-mercaptoacetyl-Phe-Tyr-amide (K(i)=41 nM) was suggested as inhibitor. Similarly, Anthrax Lethal Factor protease inhibitors were suggested in the past, for example (2R)-2-[(4-fluoro-3-methylphenyl)sulfonylamino]-N-hydroxy-2-(tetrahydro-2H-pyran-4-yl)acetamide (Xiong Y. et al, 2006, U.S. Pat. No. 7,504,425 B2), as well as a compound named NSC12155 (Panchal R. G. et al. 2004), and substrate derived hydroxamates with Ki of up to 1 nM (In-2-LF) (Tonello F. et all 2002). A peptide derived from the substrate (MLARRKKVYPYPMEPTIAEG-amide) was found to inhibit LF with a Ki of 1 nM as well (Turk B. E. et al. 2002). No inhibitor of LF reached a clinical development stage so far. Side effects of some inhibitors are known, comprising, for example, the inhibition of endogenous furin in parallel to LF. In the laboratory, Nrp599 can be inhibited with EDTA and 1,10-phenanthroline (Chung et al. 2006), but further, therapeutically acceptable Nrp599 inhibitors are not published up to now.

Several inhibitors of other members of the thermolysin family were described earlier. Phosphoramidon is produced by the Bacterium Streptomyces tanashiensis, and Talopeptin is derived from it. Phosphonomadites termed ZG^{p}LL and ZF^{p}LA display inhibitory constants in the low nanomolar range (Holden et al. 1987) with respect to thermolysin.

Khan et al described 1β-d-arabinofuranosyl-N⁴-lauroylcytosine as potent inhibitor of thermolysin out of set of 12 inhibitors found in virtual screening run (Khan et al. 2009); and 3-Phenyl-2-(trifluoromethyl) quinazolin-4(3H)-one as most potent inhibitor out of a set of novel 38 quinazolin-4(3H)-ones (Khan et al. 2010).

Therapeutic inhibition of thermolysin activity by administering non-specific metalloproteinase inhibitors, however, is biased by negative side effects induced by collateral inhibition of essential host enzymes such as its matrix metalloproteinases. Phosphoramidon, for example, is inhibiting the endogenous protein endothelin converting enzyme (ECE). Furthermore, some of these compounds are toxic, while for others the toxicity is obviously unknown. Consequently, a strong demand for molecules capable of specifically inhibiting one microbial enzyme (Adekoya et al. 2009) or a corresponding enzyme family persists.

Streptomyces metalloproteinase inhibitor (SMPI) (Oda et al. 1979) is a known proteinaceous inhibitor of thermolysin. SMPI consists of 102 amino acids and exhibits two cystein bridges exhibiting an IC50 of 0.6 nM for Thermolysin (Hiraga et al. 1999). It was discovered, however, that SMPI is degraded by other bacterial proteases (Tsuru et al.1992), disqualifying SMPI for use as a compound for treating bacterial infections. Furthermore, its low molecular weight of ca. 12 kD means that it is rapidly cleared from the bloodstream by renal filtration.

The only other peptide inhibitor of thermolysin reported to date which specifically inhibits thermolysin-like enzymes is the insect metalloproteinase inhibitor IMPIα. It was originally discovered in and purified from the hemolymph of immunized G. mellonella larvae (Wedde et al. 1998). Its active moiety comprises 69 amino acids including intramolecular cystein bonds, and a molecular weight of 7667.7 Da. The molecule has a reported IC50 of 0.62, 0.86 and only 81.66 nM for thermolysin, bacillolysin and pseudolysin, respectively, all of which are enzymes belonging to the M4 protease family (Clermont et al. 2004). IMPIα was tested against human metallo-matrix proteases MMP1,2,3,7,8, and -9, of which only MMP1 and MMP2 showed a negligible inhibition. IMPIα was recombinantly produced in Schneider cells (Clermont et al. 2004) and later in E. coli. Although the structure of IMPI was not known until recently (Gomes-Rueth. 2011), it was suggested by homology searches that IMPIα should belong to the I8 or Ascaris family of serin protease inhibitors, despite the fact that IMPIα inhibits a metalloprotease and not a serin protease (Wedde et al., 2007). From this comparison it was further deduced that an active site loop would be present in IMPIα between aa 33-aa40, including a cleavage site between aa37 (Asparagine) and aa38 (Isoleucin). Other known protein inhibitors of metalloproteinases do not inhibit proteinases of the M4 protease family.

In summary, most known inhibitors of members of the M4 protease family are too toxic, unspecific, unstable or difficult to manufacture or express, or require a different specificity profile against metalloproteases.

### SUMMARY OF THE INVENTION

It is therefore an object of the present invention to provide inhibitors of M4 protease family members with an increased potency for specific M4 proteases, a selected if not engineered specificity profile including the potential requirement for a broad band metzincin inhibitior. Furthermore, higher stability, lower toxicity or more efficient manufacturing or expression profile is requested.

The object underlying the invention is accomplished by a polypeptide according to claim 1 and other independent claims. The claims depending on one or more independent claims are concerning specific embodiment of the invention.

The polypeptide is a polypeptide having at least 70% homology, in particular 80%, 90% or 95% homology to the polypeptide of SEQ ID NO:2 representing the wild-type of the protein insect metalloproteinase inhibitor IMPIα and having at least one mutation at position 35, 36 and/or 39 of the amino acid sequence of IMPIα wherein
the nonpolar amino acid isoleucine at position 35 of IMPIα is replaced either by a nonpolar amino acid selected from the group consisting of leucine, methionine and phenylalanine or by polar amino acid selected from the group consisting of cysteine, asparagine, glutamine, histidine, lysine and arginine; and/or
the nonpolar amino acid isoleucine at position 36 of IMPIα is replaced either by a nonpolar amino acid selected from the group consisting of valine, phenylalanine and tryptophan or by polar amino acid selected from the group consisting of tyrosine, serine, threonine, asparagine, glutamine, histidine, lysine and arginine; and/or
the polar amino acid position 39 of IMPIα is replaced either by the nonpolar amino acid valine or by the polar amino acids histidine or lysine.

The polypeptide of the invention shows in particular an IC₅₀ value to thermolysine of less than the IC₅₀ value of IMPIα.

In an embodiment of the invention the polypeptide of the invention has the amino acid sequences of SEQ ID NOs:10, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84.

In an another embodiment of the invention the polypeptide of the invention may comprise chemical modifications in the side chain or at the N and/or C terminal for improving biological or chemical properties such as bio availability, stability, effectivity. The modification may also provide for a detectable label, for example a chem-iluminescent structural element, one or more radioactive isotopes in one or more side chains of an amino acid in the polypeptide, an enzyme which is able to generate a colour reaction and the like.

Subject matter of the present invention is also a fusion polypeptide comprising the polypeptide of the invention and at least one polypeptide having a physiological function in particular an antibody or antibody fragment, scaffolds such as lipocalin, ankyrin, fibronectin, transferrin, tetranectin, adnectin, albumin, uteroglobin, or protein A, functional peptides such as transferrin, peptides useful for diagnostic applications, such as green fluorescent protein (GFP), or peptide tags enabling immobilization on technical surfaces, such as hexahistidine, or glutathione-S-transferase (GST).

In an embodiment of the invention the polypeptide of the invention has the amino acid sequences of SEQ ID NOs:6, 8, 12, 86, 88, 90, 92.

There are three superfamilies (cytosolic, mitochondrial, and MAPEG) of GSTs: while classes from the cytosolic superfamily of GSTs possess more than 40% sequence homology, those from other classes may have less than 25%. Cytosolic GSTs are divided into 13 classes based upon their structure: alpha, beta, delta, epsilon, zeta, theta, mu, nu, pi, sigma, tau, phi, and omega. Mitochondrial GSTs are in class kappa. The MAPEG superfamily of microsomal GSTs consists of subgroups designated I-IV, between which amino acid sequences share less than 20% identity. Human cytosolic GSTs belong to the alpha, zeta, theta, mu, pi, sigma, and omega classes, while six isozymes belonging to classes I, II, and IV of the MAPEG superfamily are known to exist:

| GST Class | Homo sapiens GST Class Members |
|---|---|
| Alpha | GSTA1, GSTA2, GSTA3, GSTA4, GSTA5 |
| Kappa | GSTK1 |
| Mu | GSTM1, GSTM1L (RNAi), GSTM2, GSTM3, GSTM4, |
| | GSTM5 |
| Omega | GSTO1, GSTO2 |
| Pi | GSTP1 |
| Theta | GSTT1, GSTT2, GSTT4 |
| Zeta | GSTZ1 (aka GSTZ1 MAAI—Maleylacetoacetate |
| | isomerase) |
| Microsomal | MGST1, MGST2, MGST3 |

In a further embodiment of the invention the fusion polypeptide of the invention is linked by a peptide of 1 to 100 amino acids in length to the polypeptide of the invention. Alternatively polypeptide of the invention can be conjugated by a chemical linking group with a polypeptide having a physiological function to yield the fusion polypeptide of the invention.

Polypeptides or proteins can be manufactured by chemical methods, such as solid phase syntheses or by means of genetic engineering by means of utilizing coding polynucletides in a suitable expression system. Subject matter of the present invention is also a polynucleotide coding for the polypeptide of the invention or the fusion polypeptide of the invention. The polynucleotide of the invention may be operably linked to a heterologous promoter.

Subject matter of the present invention is also a pharmaceutical composition comprising the polypeptide of the invention and/or the fusion polypeptide of the invention or their derivatives and/or a nucleic acid comprising a section coding for the polypeptide of invention.

The polypeptide of the invention or the fusion polypeptide of the invention can be used in the treatment of an animal or human infected by microorganisms secreting bacterial toxins of the M4 or Metzincin family of metalloproteinases, in particular thermolysine, aureolysin, bacillolysin, pseudolysin, vibriolysin or anthrax npr599.

The polypeptide and/or the fusion polypeptide of the invention can be used for detection of the presence or activity of proteases belonging to the M4 family in a sample.

The invention is based on the result that mutations between amino acid 35 and 39 of IMPIα (SEQ ID NOs:10, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84) could be used to alter systematically the specificity profile and potency related to specifically inhibiting members of the thermolysin M4 protease family. Accordingly, the polypeptide IMPIα of the invention inhibit further proteases of the thermolysin M4 protease family, particular thermolysine, aureolysin, bacillolysin, pseudolysin, vibriolysin or anthrax npr599, and that the mutein IMPIα exhibit different inhibitory constants with respect to the various proteases.

It was further found that all polypeptides of the invention and wtIMPIα consisting of SEQ ID NO:2 can all be produced very effectively employing the same recombinant process in bacteria, particularly in E. coli, in high quantities, high quality and without requiring refolding. In case of wtIMPIα, the DNA is including an oligonucleotide as SEQ ID NO:1. This discovery is surprising because wtIMPIα and all the polypeptide of the invention contain five cystein bridges each. Proteins or peptides containing many cystein bridges are usually considered as difficult or impossible to express in any bacteria, including E. coli., and published protocols reveal low production efficacy and requirement for cooling (see, for example, Comis Ruets, 2011) while according to the method of invention wtIMPIα and the polypeptide of the invention can be produced without requiring cooling of the fermenter during the protein expression phase, and without requiring protease inhibitors to be added to protect the protein product from degradation.

As used herein, the following terms have the following meanings unless expressly stated to the contrary.

The term “wtIMPIα” refers to a protein with an amino acid sequence as in SEQ ID NO:2 and consists of the N-terminal fragment of the full length IMPI molecule which is endogenously cleaved from the larger precursor molecule IMPI.

The term “wtIMPIβ” refers to a protein with an amino acid sequence as in SEQ ID NO:4 and consists of the C-terminal fragment of the full length IMPI molecule which is endogenously cleaved from the larger precursor molecule IMPI.

The term “loop mutein IMPIα” comprises all proteins comprising an amino acid sequence identical to SEQ ID NO:2 (wtIMPIα), with the exception of the amino acid stretch 35 to 39, and in particular with exception of the amino acid stretch 35, 36, or 39, in which at least one amino acid is exchanged, added, or deleted. By this definition “Loop mutein IMPIα” includes, for example, amino acid sequence SEQ ID NOs:18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, in which one amino acid at position 35, 36, or 39 is exchanged. In SEQ ID NO:10 two additional amino acids at positions 37 and 38 are exchanged.

The term “mtIMPIα” or “Mutein IMPIα” is the polypeptide of the invention and includes all recombinant or synthetic proteins with an amino acid sequence which is at least 70%, especially 80% 90%, 95% homologous, but not identical to SEQ ID NO: 2 and which inhibits thermolysin or another protease of the M4 protease family with an IC₅₀<1000 nM, especially IC₅₀<100 nM, in particular those which can be expressed in E. coli and especially those including proteins of the group “loop mutein IMPIα” and “structure variants of IMPIα”. mtIMPIα include inserted, substituted or deleted residues, including N-terminal and C-terminal and intrasequence deletions, provided the selected mutations preserve the biological activity of the Mutein IMPIα.

The term “M4 family of proteases” refers to the definition given in the MEROPS online database of peptidases (Wellcome Trust Sanger Institute, Cambridge, UK), and described also by Barret et al. (2004).

The term “biologically active” as used herein refers to wtIMPIα or the polypeptide of the invention demonstrating inhibition of thermolysin or another protease of the M4 protease family with an IC₅₀<1000 nM especially IC₅₀<100 nM or better.

The term “IMPIα family” alone shall include biologically active mtIMPIα and wtIMPIα

### BRIEF DESCRIPTION OF THE FIGURES

FIG. 1: Vectormap of the hybridplasmid pET41IMPIα. Total mRNA was isolated from immunchallenged Galleria melonella and subsequently used as template for the amplification of DNA coding for IMPIα. The amplicon was then cloned into the PshA1 linearized pET-41A vector (Novagen) resulting in a GST (Glutathion-S-transferase) fusionprotein. Accurate in frame cloning of IMPIα into the vector was verified by sequencing.

FIG. 2: SDS-PAGE of the soluble cytoplasmatic fraction of Rosetta gami 2 three hours after induction of the T7-Expression system. The overexpressed fusionprotein is marked by an arrow. The corresponding controls (empty pET-41 and the uninduced T7-Expression system) show no corresponding bands.

FIG. 3 HPLC UV detector data from purification run of GST-tagged IMPIα. The purification has been carried out by affinity chromatography on a HPLC, using a resin with immobilized glutathion at room temperature. The purification has been carried out by affinity chromatography on a HPLC, using a resin with immobilized glutathion at room temperature.

FIG. 4a SDS-PAGE of the purified GST-tagged IMPIα For larger amounts of mutein GST Bind Fractogel Cartriges (Merck, Darmstadt) were used, while for amounts less than 40 mg glutathione spin columns (Pierce; Thermo Scientific, Rockford) have been deployed.

FIG. 4b: SDS-PAGE after the specific cleavage of the n-terminal GST-tagged IMPI alpha by recombinant enterokinase (Merck, Darmstadt). The cleavage was carried out for 16 hours at room temperature. Untagged IMPIα is marked by an arrow.

FIG. 5: Sucessfull downstream processing was verfied by mass spectroscopy on a micrOTOF-Q mass spectrometer (Bruker Daltonics). Analysis of samples was achieved by chromatography on a reverse-phase column (Acclaim 120, C8, 3 μm, 120 Å, 2.1×150 mm; Dionex) by applying a gradient of 1-80% (1,63%/min) acetonitril in water containing 0.1 percent formic acid. By-product (peak termed 1 and 3) and main product (peak 1) show the correct mass of 7,677 kDa.

FIG. 6: wild type protein assembly of full length IMPI, including IMPIα and IMPIβ in G. melonella.

FIG. 7: inhibitory plot of wtIMPIα and phenanthronin vs. the anthrax protein nrp599 and InhA.

FIG. 8a: Multiple alignement of IMPIα homologues of Galleria melonella.

FIG. 8b: Multiple alignment of IMPI's found in the transcriptome of other lepidoptera species (Sequences unpublished).

FIG. 8c: Pairwise alignment of the M4-metalloprotease thermolysin from Bacillus thermoproteolyticus with the metalloprotease npr599 from Bacillus anthracis (strain A0248).

FIG. 9: The binding of the peptidic inhibitor IMPIα to the M4-metalloprotease thermolysin (Bacillus thermoproteolyticus) is depicted. Red areas represent Van der Waals contact areas between inhibitor and protease. The isoleucin (P1′)(purple) fits perfectly into the S1′ pocket which is responsible for specificity of the metalloptotease hence the inhibitor is binding in a lock and key kind of fashion.

FIG. 10: Schematic drawing of a lateral flow device for detecting protease specific activity. The principle is based on thin layer chromatography. The eluent carries ichor collected by a cotton swap via tangential flow through the device. M4-metalloproteases produced by pathogens therefore also migrate through the device, until they get bound to IMPI muteins exhibiting certain specificity for different metalloproteases. The different muteins are bound at concentrated spots on the solid phase of the device, so that different metalloproteases can be clearly identified. Spots are made visible afterwards by detection antibodies present in the mobile phase.

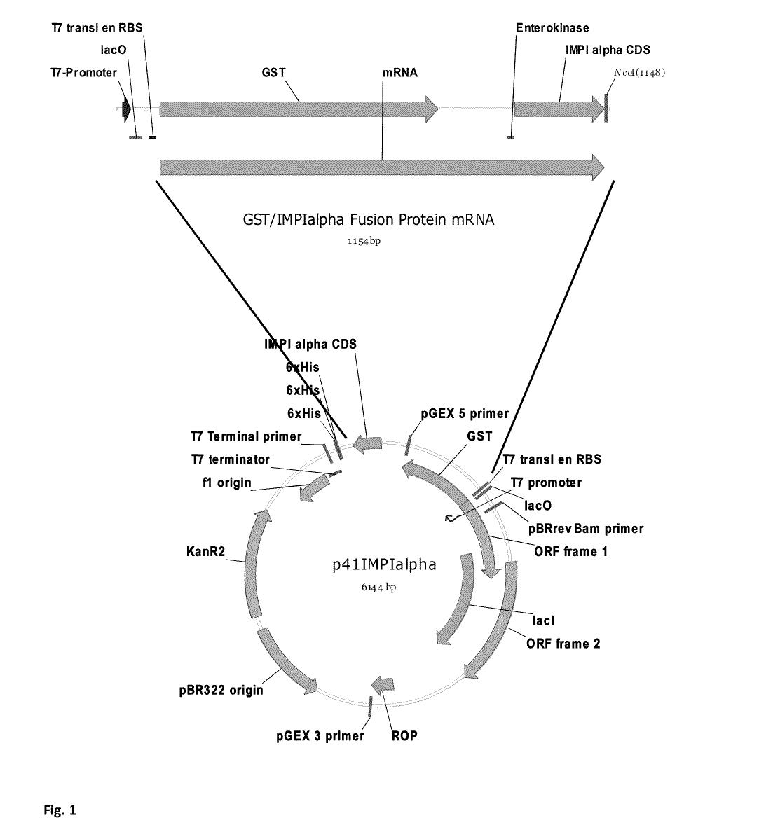

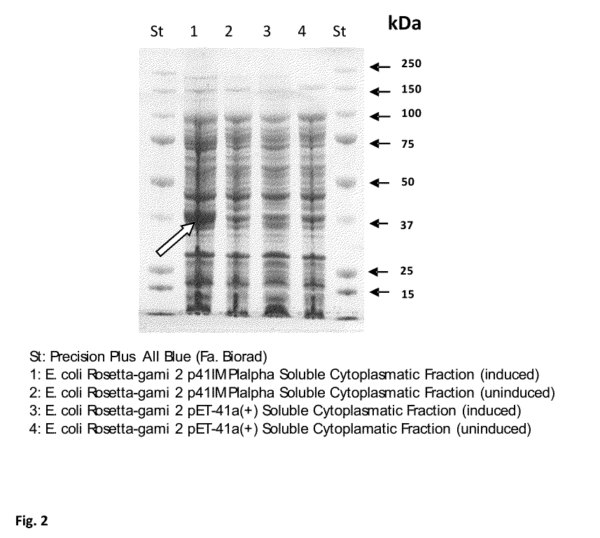

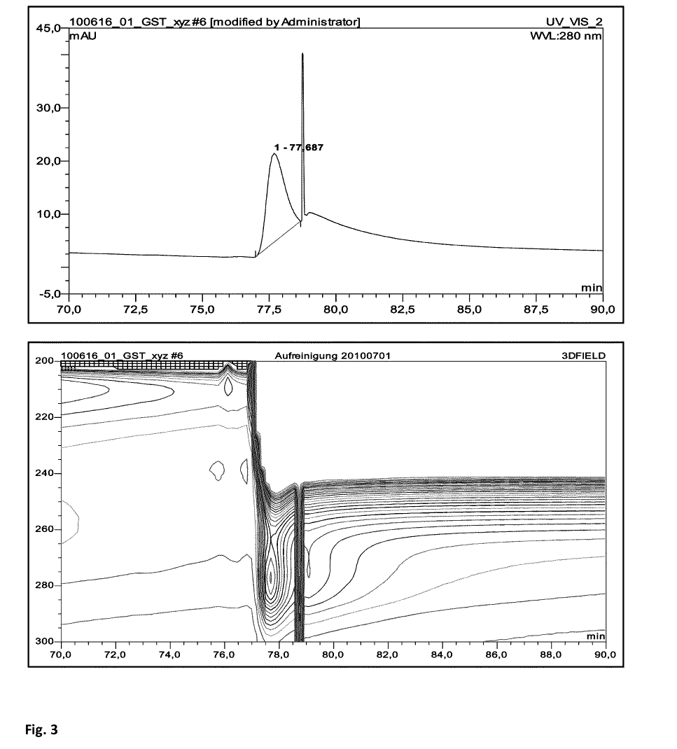

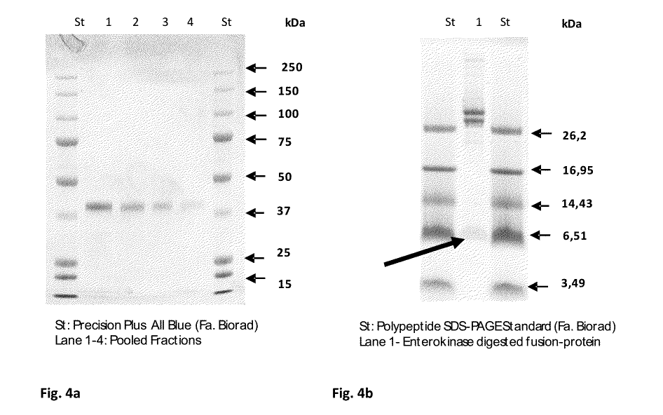

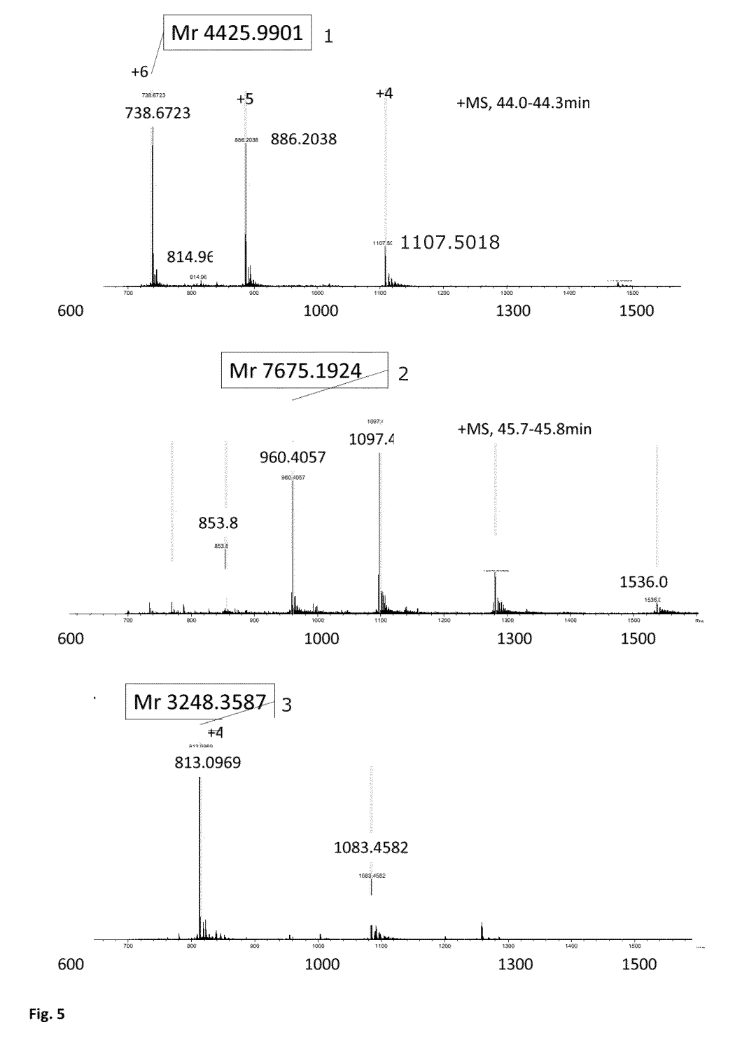

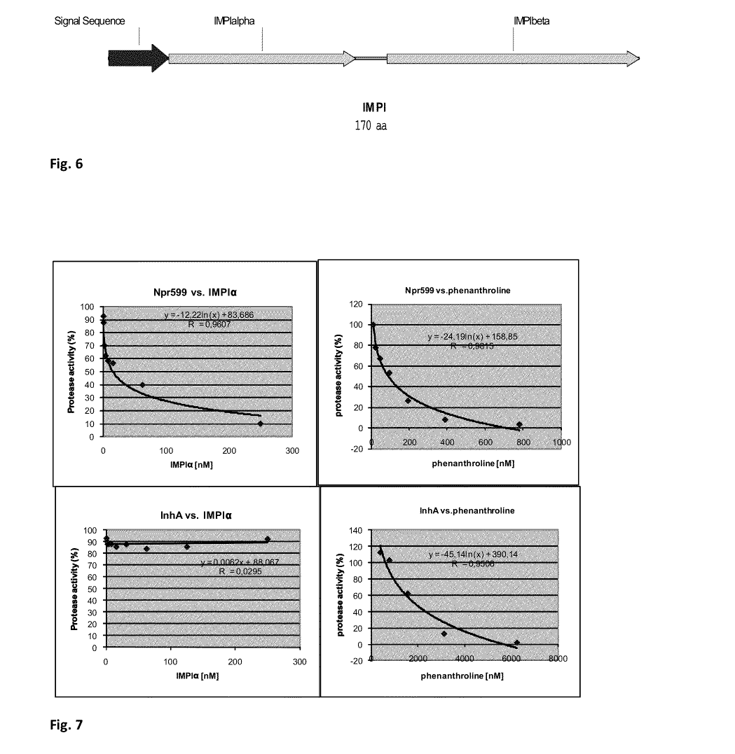

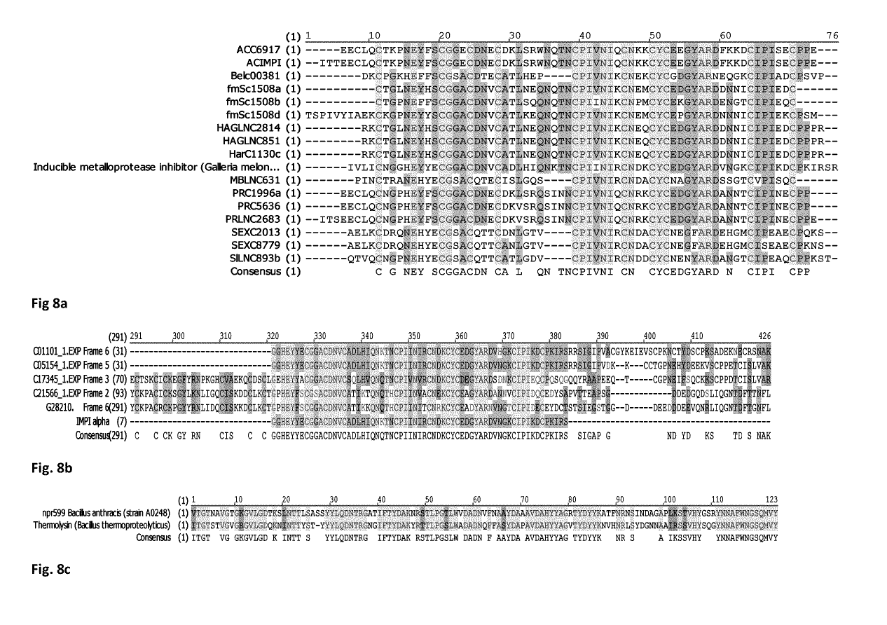

Table 1: Overview of amino acid exchanges and respective functionality for Thermolysine and Pseudolysine, and respective ratio of IC₅₀ values. The ratio changes mean that an inhibitor with designed ratio of IC₅₀s with respect to different proteases can be created.

Table 2: Detailed experimental parameters and results for inhibition of Thermolysine.

### DETAILED DESCRIPTION OF THE INVENTION

In one aspect of the invention, biologically active loop polypeptide of the invention polypeptides are provided which exhibit a specificity profile different from the wtIMPIα profile for inhibiting members of the M4 protease family. The fact that the loop polypeptide of the invention are biologically active at all is surprising because the loop amino acids were particularly well conserved during evolution. It is even more surprising that altering a specific loop amino acid could increase the binding constant for thermolysin or pseudolysin, or even both.

New ways of improving the inhibitory constant by substituting I36 (Isoleucine) of wtIMPIα have been developed, which is surprising since it was assumed that at this position 36 isoleucine would already exhibit a perfect profile for a C-C interaction with amino acids H146 (histidine) and T157 (tyrosine) of the peptidase, and therefore no attempts were reported to design IMPI mutants or small molecules with the goal to strengthen this interaction further. The inventors found, however, that a long aliphatic side chain of an amino acid or modified amino acid at this position, preferably with an elevated pKa of 12 or higher, improves the inhibitory constant substantially. Examples of well suited amino acids comprise K, M, H, and in particular R.

Surprisingly it was found that further substitutions, for example those listed in the ensuing paragraph, could increase the potency of the inhibitor (Tables 1, 2):

Loop the polypeptide of the invention molecules exhibiting improved inhibition of Thermolysin comprise I35L, I36F, K, R, V, Y, Q, D, H, M, T, or W, R39K or V.

Loop the polypeptide of the invention molecules exhibiting improved inhibition of Pseudolysin comprise I35L or F, M, W or Y, I36R, Q, or M, R39K and A.

Loop polypeptide of the invention molecules exhibiting improved inhibition of both, Thermolysin and Pseudolysin in parallel comprise I35L, I36R, Q, or M, R39K.

Loop polypeptide of the invention exhibiting equal potency against both, Thermolysin and Pseudolysin, comprise I35L, or W.

This result is surprising because there no reasonable expectation of success if one amino acid is altered close to an area associated with the function of the respective protein. In particular this result is surprising considering the general assumption that a peptidic inhibitor of an enzyme should contain hydrophobic amino acids binding to the enzyme close to its hydrophobic active center (Handbook of proteolytic Enzymes 2013, Biela et al. 2013). The results show, however, that hydrophobicity of the amino acids in the polypeptide of the invention is not the dominant factor for the inhibition.

It is understood that combinations of single site mutations could alter the properties of the polypeptide of the invention even further, and further increase the potency of the inhibitor.

The polypeptide of the invention may be obtained by known techniques for directed evolution as described by Arnold et al., 2003a and Arnold et al., 2003b, or may further be obtained by knowledge based engineering of the respective amino acid sequence, including application of rules for conservative or non-conservative amino acid replacement considering size, charge, polarity and other biochemical parameters of the amino acids as know by one of ordinary skill in the art by the teaching and principles disclosed in U.S. Pat. Nos. 7,732,587 and 4,554,101, or by using suitable computer programs calculating or estimating the binding energy of the Mutant IMPIα and the selected target proteases, by assessing homologies to other proteins or the antigenicity of the protein sequence, and introducing the calculated, modified gene sequence into the bacterial genome by standard molecular biotechnology methods. Amino acid deletions within any IMPIα and its muteins may be made in regions outside the loop region (aa 35 to 39 of SEQ ID NO:2), where they are supposed to not directly interact with the target protease. Amino acid deletions may also be made within the loop region, where they are supposed to more likely modify the biological activity.

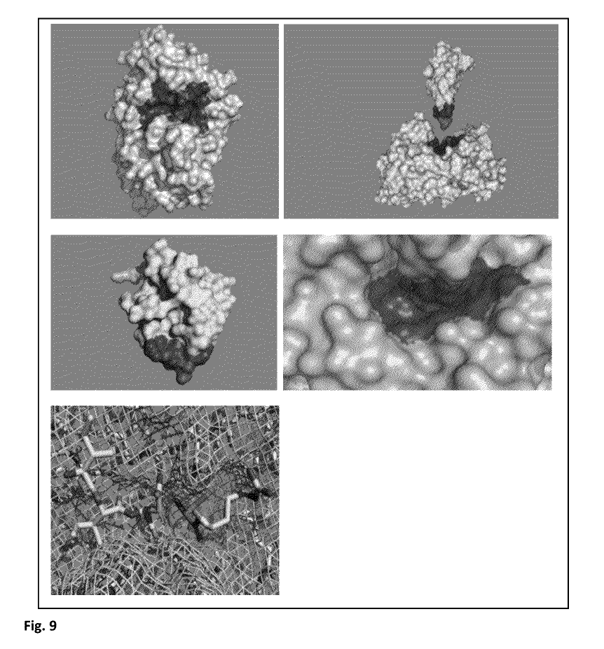

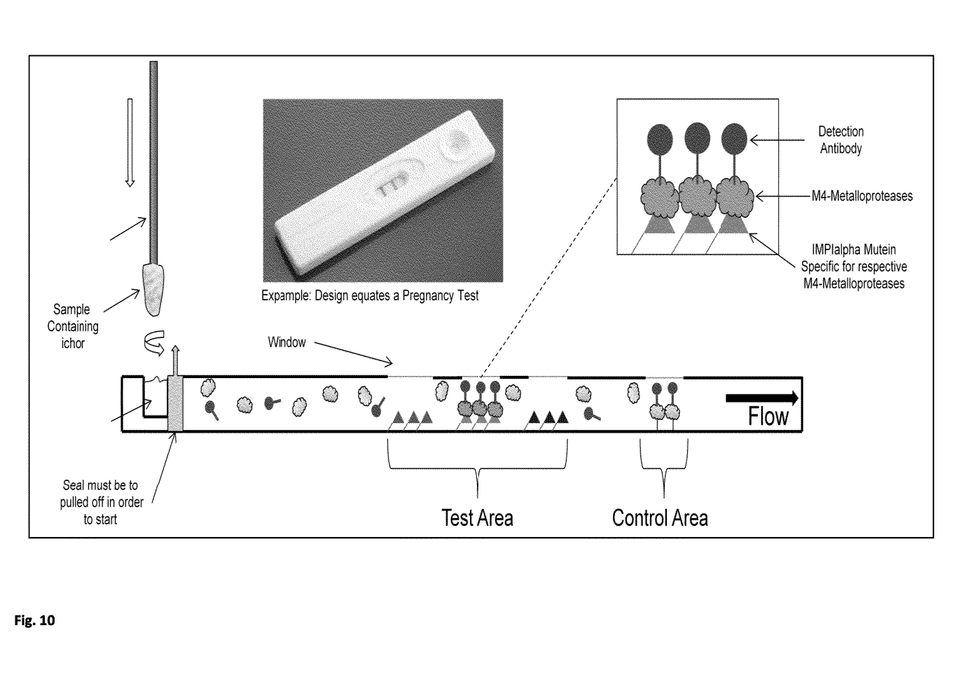

Comparison of wtIMPIα with similar proteins from other species, such as SEQ ID NO:95 from Solenopsis, and 23 other insect related aa sequences depicted in FIG. 9 reveals that the loop region (aa 33 to 41 in SEQ ID NO:2, and in particular in region 35 to 39) is highly conserved with the exception of aa39 (R). It is thus surprising that Loop Mutein IMPIα, for example, SEQ ID NOs:10, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84 also exhibit a strong, undexpected thermolysin inhibiting activity.

In another aspect of the present invention it has been found that wtIMPIα and Mutein IMPIα, and in particular Loop polypeptide of the invention can be recombinantly produced in bacterial cells under conditions identified by the inventors, especially at room temperature and up to 37° C., under conditions which do not require cooling of the fermenter or refolding of the protein produced. The method according to the invention, equipment and reagents used are described in detail in example 2 below. It should be understood, however, that modifications in concentrations of reagents used, and modifications in the genetic configuration of the producer organism (E. coli) could be made without affecting the output of correctly folded protein. For example, E. coli could be replaced by other bacteria as production host, additional plasmids or gene stretches with additional function could be added to the producer bacteria. Furthermore, medium components could be provided in different order or concentrations, and incubation times could be varied. It should be understood that those modifications which influence the production efficacy of biologically active Mutein or wtIMPIα negatively by 50% or less or even positively are considered within the scope of the invention.

The invention contemplates further modifications of elements of the expression system inasmuch as the expression of a functional Mutein or wtIMPIα without the need for refolding is maintained to at least 50% compared to the system disclosed in Example 3 and 4, and no cooling of the fermenter is required. For example, other expression vectors than pET-41a(+) may be used, which may not be linked to a fusion tag, or may be linked to a fusion tag other than GST, His-tag; and other promoters than T7, and may also include further regulators or other fusion elements. Depending on the operons employed, IPTG or other inducers may be employed. Other restriction sites than PshAI may be used in pET-41a(+) or in other vectors, for example XhoI, EcoR I, BamH 1. Other E. coli strains than Rosetta-gami 2 pLysS may be used, with or without additional t-RNA genes or ahp C mutation, and other bacteria than E. coli may also be employed.

Further modifications may relate to the downstream process steps which are known to the one skilled in the art, including but not limited, to incubation times, reagents and their concentrations and disposables used, for example capture resins and chromatography equipment and separation media for purification.

Recombinant production of Mutein or wtIMPIα in bacteria is very cost effective compared to all other production methods for proteins. Cloning and the subsequent development of a cell line producing the peptide are faster and less costly with bacteria as, for example, with mammalian or insect cells. During production, the amount of protein produced per volume fermentation broth is much higher compared to eukaryotic cell cultures. Consequently there is a strong demand for methods of recombinant protein producing in bacteria, especially in E. coli since they are a proven host frequently used for protein production. It is known that wtIMPIα retains its biological activity after deglycosylation (Wedde et al. 1998); therefore bacterially produced Mutein or wtIMPIα lacking glycosylation are biologically active once folded correctly.

The skilled artisan would expect, however, that a protein like Mutein or wtIMPIα exhibiting 5 cystein bridges would not be correctly folded and thus be accumulated almost entirely in inclusion bodies of the E. coli host. While several processes were developed earlier to isolate and dissolve inclusion bodies, and to denature its protein content using chaotropic salts like urea or guanidylchloride, and to withdraw the salts again in an ensuing step, for example by dialysis, to enable refolding of the protein in a kinetically controlled manner. However, the success rate of refolding proteins with multiple cystein bridges, such as as IMPIα, is usually low, and even today time consuming and resource consuming trial and error methods are required to find adequate refolding conditions. It is generally not yet possible to predict or experimentally determine suitable refolding conditions.

It is thus surprising that, without any known exception, a family of cystein rich proteins such as Mutein or wtIMPIα with a complex tertiary structure can be recombinantly produced in bacteria by the method of invention and without the need for refolding steps, in particular that the production can take place without cooling of the fermenter, the latter being a common measure to reduce the protein production rate of the bacteria and thereby to reduce the ratio of incorrectly folded protein vs. correctly folded protein.

In another aspect of the invention, the use of Mutein or wtIMPIα, and in particular of wtIMPIα is provided for inhibition of the anthrax protease nrp599. Current approaches to treat Anthrax include vaccination such as BioThrax (Emergent Biosolutions, Rockville Md., U.S.A) for disease prevention, or antibiotics such as Ciprofloxacin or Doxycyclin (National Terror Alert Response Center). While the vaccine is only approved for preventive use, it takes several days until antibiotics exert effects on bacteria to an extent that the symptoms caused by their virulence factors are reduced. Compounds neutralizing virulence factors were developed recently and are generally well known in the state of the art and for anthrax most compounds targeting virulence factors developed so far target LF. However, no specific inhibitor of nrp599 was reported so far, although recent evidence shows that nrp599 is one of the major anthrax virulence factors inducing hemorrhagic bleeding, for example (Chung et al., 2006) by targeting the fibrinolytic system (Chung et al, 2011), and that it also degrades the van Willebrand-Factor and thereby interferes with the coagulation cascade (Chung et al., 2008).

US 2004/018193 teaches the concurrent or offset administration of both, antibiotics and antigenic compounds, in particular to treat anthrax infected humans. The authors provide several examples of protease inhibitors including hypothetical antibodies, but they do not disclose nrp599 as a target.

Surprisingly it was found that polypeptides of the invention and in particular wt IMPIα is a potent and specific inhibitor of nrp599, despite the fact that bacillus anthracis is not known to infect insects. It was found that wtIMPIα inhibits npr599 at nanomolar concentrations.

To treat anthrax infection or symptoms caused by anthrax toxins, the polypeptide of the invention and in particular wt IMPIα may be combined with antibiotics, in particular Ciprofloxacin or Doxycyclin, with antimicrobial peptides, with other inhibitors of bacterial proteases, or with compounds improving the physiological state of the infected patient.

The polypeptide of the invention may also be produced effectively in recombinant cell lines and tissue, including mammalian and insect expression systems, plant and bacterial systems; or synthetically.

Mutein and wtIMPIα each may typically be isolated and purified to be free of other proteins and protein fragments. Preferably, Mutein and wtIMPIα is about 80% free of other proteins which may be present due to the production technique used in the manufacturing process. More preferably, the polypeptide of the invention or wtIMPIα is about 90% free of other proteins, particularly preferably about 95% free of other proteins, and most preferably about >98% free of other proteins. It will be appreciated, however, that the desired protein may be combined with other active ingredients, chemical compositions and/or suitable pharmaceutical formulation materials prior to administration as medication, as described in further detail below.

The biological activity of Mutein or wtIMPIα can be assessed in vitro by means of assays, such as measuring binding of the polypeptide of the invention or wtIMPIα to a protease immobilized on coated glass carrier used for Plasmon resonance spectroscopy. Alternatively, the polypeptide of the invention or wtIMPIα can be immobilized on the glass carrier and the protease could be added to start the assay. This assay is able to measure on- and off-rates for the respective interaction.

The biological activity can also be tested by means of an Parallel Line assay, for example in a microtiter plate format, and by detecting a coloration effect or by measuring changes in the fluorescence emitted upon excitation with a suitable light source.

Amino acid sequence additions may further include N- or C-terminal fusions ranging in length from one residue to 150 or more residues, as well as internal intrasequence insertions of single or multiple amino acid residues into Mutein or wtIMPIα ranging typically from about 1 to 10 amino acids, more typically from 1 to 5 amino acids, and most typical from 1 to 3 amino acids. N-terminal additions include the addition of a methionine or an additional amino acid residue or sequence. Another example of N-terminal addition includes the fusion of a signal sequence—provided the signal sequence SEQ ID NO:94 is not bluntly added to wtIMPIα of SEQ ID NO:2.

Further examples for additions to Mutein or wtIMPIα comprise fusions with antibodies (Abs) or antibody fragments. Ab fragments may comprise for example scFAb, scFv fragments, or single domain antibodies, or antibodies of camelide (hcIG) or shark (IgNAR) origin, or their respective VHH domains. The fusion may serve to provide a targeting moiety (the CDR domains) fused to to Mutein or wtIMPIα so that the fusion protein could be accumulated and enriched at surface displaying antigens specific for the antibody fragments. Another motivation for fusing Mutein or wtIMPIα to antibodies may be to benefit from the dimerization properties of the constant antibody regions to obtain a bivalent form of Mutein or wtIMPIα, in a way similar to Etanercept (Enbrel) in which 2 TNF receptor fragments are fused to the constant domains of an antibody. In addition, such bivalent antibody chimera is sufficiently large to escape from rapid renal clearance, for which a size exclusion barrier of roughly 65 kDa exists, so that the lifetime of the fusion protein in the patient body is strongly prolonged. Additions may also comprise other scaffolds derived, for example, from lipocalin, ankyrin, fibronectin, transferrin, tetranectin, adnectin, albumin, uteroglobin, or protein A. Other additions contemplated comprise functional peptides, for example transferrin enabling the fusion protein to cross the blood brain barrier, a property potentially useful for inhibiting bacterial toxins accumulated in the brain or spinal cord, or additions useful for diagnostic applications, such as green fluorescent protein (GFP) enabling fluorescence detection, or peptide tags enabling immobilization on technical surfaces

Specifically the polypeptide of the invention described herein may involve addition or deletion of or substitution with a non-native amino acid at the N- or C-terminus or at any site of the protein that is modified by the addition of an N-linked or O-linked carbohydrate. A cystein, for example, may be added for linking a water soluble polymer such as polyethylene glycol, or other amino acids like lysine, cysteine, histidine, arginine, asparaginic acid, glutamic acid, serine, threonine, or tyrosin could also be used for coupling polymers to the peptide. Another example is the insertion of tripeptide sequences NXT or NXS or fragments thereof with X designating any amino acid except P, which may be recognized by a cellular enzyme adding glycosylation elements. Suitable, clinically acceptable, water soluble polymers include polyethylenglycol (PEG) and polysialic acid (PSA).

Another mutation or addition may include tags, in particular peptide tags, such as hexa-histidine which can be used to facilitate binding, for example, to a moiety of a purification column. These tags may comprise a peptide sequence serving as a substrate for a protease, so that the tag can be cleaved in purpose once it is not required anymore.

Further contemplated are Mutein or wtIMPIα to which chemical compounds or nano-particles are added. This can be achieved for example by employing the enzyme O-6-Alkylguanin-alkyltransferase (AGT) or derivatives thereof to Mutein or wtIMPIα, or to a peptide tag. Other examples comprise conjugation to a cysteine being introduced by a mutation (Jagath et al. 2008), or to carbohydrate moieties (Fischer-Durand et al. 2010). Chemical compounds may include for example labeling moieties, in particular for in vivo imaging applications, targeting moieties such as receptor ligands to enrich the conjugated IMPIα family members in specific organs. Another compound suited to immobilize conjugated IMPIα family members on a surface or particle is biotin, for example for diagnostic purposes. Nanoparticles such as quantum dots may be added for in vivo detection or imaging applications. Dendrimers or others particles increasing the size of the conjugate may be added to prevent the conjugate from cleared rapidly from the blood stream by the kidneys.

Modifications to the peptide backbone are also contemplated under the invention, such as beta peptides, where the amino group is attached to the beta carbon of the respective amino acid instead of the alpha carbon, rendering them invulnerable to proteases. Other modifications of the peptide backbone are possible, such as alkylation of the amid nitrogen and bioisosteric replacement of amide groups.

The present invention further provides polynucleotides which contain nucleotide sequences encoding Mutein or wtIMPIα, provided the polynucleotide is not identical to the complete wild type sequence SEQ ID NO:1, or SEQ ID NO:1 directly followed by SEQ ID NO:3. Further are considered nucleotide sequences in which oligonucleotide stretches are inserted which do not code for wtIMPIα. Based upon the present description and using the universal codon table, one of ordinary skill in the art can determine all of the nucleic acid sequences which encode Mutein or wtIMPIα. Presently preferred nucleic acid sequences include those encoding SEQ ID NOs:9, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83.

Chemical synthesis of Mutein or wtIMPIα is also contemplated, and is the obligatory route for synthesizing modified Mutein or wtIMPIα containing non-natural amino acids or backbone modifications. While chemical synthesis was traditionally regarded as being too expensive for larger peptides, recent advances in synthesizing technology like microwave assisted synthesis have recently shifted this limit towards larger proteins. Recent approaches also solve the issue of correct configuration of currently up to 3 cystein bridges by using several distinct protective groups in parallel.

While the invention contemplates the use of E. coli for producing Mutein or wtIMPIα with high quality and efficiency, other hosts may also be used for recombinantly producing Mutein or wtIMPIα, including other strains of E. coli, other bacteria like bacillus megaterium, fungal systems like yeast, pichia, or hansenula, eukaryotic cells like protozoa, insect cell lines like Schneider T7 or Sf9 mammalian animal cell lines like CHO, human cell lines like HEK293, or transgenic animals like goats, or plants like tobacco.

Mutein or wtIMPIα or modified versions thereof may be combined with ingredients to form a pharmaceutical composition. The pharmaceutical composition may include water and salts at physiological concentrations, solubilizing or dispersing agents, or anti-oxidant, or particles forming micelles, such as liposomes. Liposomes may also contain baIMPIα or modified versions thereof internally. This pharmaceutical composition may be filled in a glass or plastic vial, or in a syringe. The pharmaceutical composition may also contain additives supporting drying or freeze-drying of the pharmaceutical composition, for example cyclodextrins or saccharides, in particular disaccharides.

Mutein or wtIMPIα or modified versions thereof may be used as a medicine, in particular as a medicine for parenteral injection, to treat diseases in an animal, in particular in a mammal or bird, or in a human, and which are related to the activity of proteases from the M4 family. Such disease could be, for example, a bacterial infection, in particular infection by staphylococcus aureus, in particular multi-resistant staphylococcus aureus, bacillus anthracis, pseudomonas aeruginosa, helicobacter pylori, vibrio cholerae, or legionella pneumophilia. Such a disease could further be systemic inflammatory response syndrome (SIRS), or sepsis.

Mutein or wtIMPIα or modified versions thereof may be administered parenterally, orally, or topically using suitable pharmaceutical compositions, or attached to a patch or wound debridement from where the medication elutes into a wound of the patient.

Mutein or wtIMPIα or modified versions thereof may be administered in combination with other treatments, in parallel or staggered, in particular with other inhibitors of virulence factors, antibiotics, or antimicrobial peptides.

Further contemplated are RNA molecules whose sequences comprise RNA sequences of Mutein or wtIMPIα, in particular sequences like SEQ ID NOs:1, 5, 9, 11, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, and which can be transcribed into Mutein or wtIMPIα protein in the target organism. Such RNA molecules may be administered in combination with transfection agent like liposomes or PEI.

The invention also contemplates the application of Mutein or wtIMPIα in devices for diagnostic purposes. In several cases it is beneficial to determine the protease activity in biological fluids, and in particular the protease activity profile if several of them are present.

For fluids contaminated with bacteria determining the protease activity would provide information not only about the presence of bacteria, but also of their activity and relative concentrations.

In wounds, and especially in slowly healing or chronic wounds, determining the protease activity profile also is of interest since the physiological stages of wound healing are characterized also by a specific evolution of protease activity during healing. It is even more important to distinguish between the activity of endogenous host proteases and the activity of bacterial proteases.

PCR or real time PCR assays do not provide sufficient information because these tests quantify the concentration of particular bacteria present, but they are not suited to test for the activity of the bacteria.

Currently laboratory tests for protease activity exist where the cleaved protease substrate becomes fluorigenic. Many of these tests determine overall protease presence; some of them are able to profile the protease concentrations (Chen et al. 2013). Currently the only Point-of-Care test for protease activity, Woundchek® manufactured by Systagenix, (Gatwick, Groβbritannien) measures overall protease activity, but cannot quantify the concentration of each protease species separately (Strohal et al. 2012).

A diagnostic method according to the invention solves this issue. Mutein or wtIMPIα is used therein to bind M4 proteases specifically. Different concentrations of proteases can be determined when more than one type of Mutein or wtIMPIα is used. The difference in binding strength between the polypeptide of the invention or wtIMPIα is then used to calculate differences in concentrations of Mutein or wtIMPIα.

A preferred embodiment of the diagnostic method uses a device similar to Lateral Flow tests known in the state of the art; in which a membrane strip (suagfähig) is used to take up the fluid sample and to transport the sample across the membrane by capillary forces. In a preferred embodiment, the membrane consists of nitrocellulose or nitrocellulose endorsed by nylon fibres or tissue. At a particular location on that strip, Mutein or wtIMPIα are immobilized so that at least part of the sample fluid passes across this location during flow. Proteases present in the sample are captured by the immobilized Mutein or wtIMPIα. In a second step one further solution containing detection capture molecules targeted against different binding epitopes of the proteases are added. These detection capture molecules are prepared to deliver a detection signal, which can be, for example, chromophoric, fluorescent, or electrochemical by means of a direct or enzymatic label. It is possible to increase signal strength by secondary capture molecules targeting the detection capture molecule. In a preferred embodiment, at least one kind of capture molecule is an antibody. A particular advantage of the diagnostic method according to the invention is that several proteases can be distinguished by means of specific binding to specific locations where different Mutein or wtIMPIα are immobilized, while only a single sort of antibody is required. Furthermore, a control area is present at a different location on the membrane strip where detection capture molecules or secondary capture molecules bind to even if no proteases are present in the sample. In a preferred embodiment, the capture functionality in the control area is provided by immobilized proteases or immobilized detection capture molecules.

In a different embodiment, capture molecules other than Mutein or wtIMPIα, in particular antibodies are immobilized on the membrane strip and the proteases are added as detection capture molecule, in particular in combination with secondary capture molecules. This embodiment also requires a contral area containing immobilized proteases or Mutein or wtIMPIα.

The assessment of the protease content is made by visual inspection or a sensor device, including CMOS based imaging sensor or scanners. In case that sensors are used, a software can be employed to calculate the true concentration ratios of the proteases from potentially overlapping binding profiles of the polypeptide of the invention or wtIMPIα if the individual binding constants or other binding parameters are known. In particular such software may use a linear equation system to calculate the individual concentrations.

In another preferred embodiment, the binding kinetics are sampled, in particular by using an imaging sensor, to determine concentrations more accurately.

The sample may be added, for example, by a pipetting device, a paper strip, a cotton swab or a needle. The device may dispose of a seal preventing uncontrolled flow across the membrane strip.

To assist in understanding the present invention, the following examples are included which describe the results of a series of experiments. The following examples relating to this invention should not, of course, be construed in specifically limiting the invention and such variations of the invention, now known or later developed, which would be within the purview of one skilled in the art are considered to fall within the scope of the present invention as hereinafter claimed.

TABLE 1

| | wt | Pos39 | | | | | | | | | | | |
|---|---|---|---|---|---|---|---|---|---|---|---|---|---|
| | (avg) | RnG | RnK | RnL | RnV | RnW | RnF | RnI | RnM | RnA | RnQ | RnT | RnY |
| IC50 mutein TLN | | 347.031 | 7.78 | 58.729 | 9.244 | 64.356 | 245.863 | 87.136 | 24.551 | 18.073 | 56.329 | | |
| IC50 wt TLN | 12.53 | 12.728 | 12.524 | 12.41 | 12.538 | 12.362 | 12.489 | 12.548 | 12.571 | 12.542 | 12.605 | | |
| IC50 mutein PLN | | 1053.848 | 11.373 | 587.631 | 139.828 | 525.002 | 95.622 | 384.625 | 85.036 | 13.284 | 105.717 | 26.032 | 10000 |
| IC50 wt PLN | 21.39 | 20.318 | 21.987 | 20.102 | 21.757 | 19.948 | 20.523 | 21.168 | 22.142 | 22.076 | 22.145 | 21.943 | 22.526 |
| ratio TLN:PLN | 0.59 | 0.33 | 0.68 | 0.10 | 0.07 | 0.12 | 2.57 | 0.23 | 0.29 | 1.36 | 0.53 | | |

| | wt | Pos38 | | | | | | | | |
|---|---|---|---|---|---|---|---|---|---|---|
| | (avg) | InF | InG | InQ | InL | InV | InH | InM | InW | InY |
| IC50 mutein TLN | | 38.331 | 144.113 | 267.172 | 92.044 | 26.282 | 336.219 | 210.619 | 571.873 | 5.625 |
| IC50 wt TLN | 12.52 | 12.571 | 12.481 | 12.614 | 12.563 | 12.563 | 12.491 | 12.478 | 12.375 | 12.553 |
| IC50 mutein PLN | | 86.095 | 484.676 | 291.099 | 273.497 | 35.181 | 777.328 | 280.957 | 128.451 | 10000 |
| IC50 wt PLN | 21.80 | 21.085 | 22.796 | 22.201 | 21.734 | 21.523 | 23.139 | 21.397 | 21.148 | 21.148 |
| ratio TLN:PLN | 0.57 | 0.45 | 0.30 | 0.92 | 0.34 | 0.75 | 0.43 | 0.75 | 4.45 | 0.00 |

| | wt | Pos37 | | | | | | | | | | | | | |
|---|---|---|---|---|---|---|---|---|---|---|---|---|---|---|---|
| | (avg) | NnC | NnD | NnE | NnG | NnH | NnQ | NnS | NnY | NnA | NnM | NnP | NnV | NnW | NnL |
| IC50 mutein TLN | | 307.492 | 1379.887 | 183.989 | 590.669 | 198.251 | 10000 | 488.91 | 179.264 | 5000 | 428.764 | 749.35 | 227.708 | 250.085 | 97.854 |
| IC50 wt TLN | 12.37 | 12.674 | 12.136 | 12.644 | 12.649 | 12.78 | 12.553 | 12.499 | 12.546 | 12.553 | 11.678 | 11.982 | 12.679 | 12.674 | 11.097 |
| IC50 mutein PLN | | 332.781 | 45.714 | 5000 | 68.213 | 174.993 | 310.997 | 176.936 | 296.224 | 5000 | 136.086 | 137.03 | 234.271 | 125.939 | 137.03 |
| IC50 wt PLN | 22.17 | 20.731 | 22.607 | 21.945 | 21.827 | 21.18 | 22.224 | 21.472 | 21.501 | 21.945 | 30.792 | 20.761 | 21.194 | 21.464 | 20.761 |
| ratio TLN:PLN | 0.56 | 0.92 | 30.19 | 0.04 | 8.66 | 1.13 | 32.15 | 2.76 | 0.61 | 1.00 | 3.15 | 5.47 | 0.97 | 1.99 | 0.71 |

| | wt | Pos36 | | | | | | | | | | | | | |
|---|---|---|---|---|---|---|---|---|---|---|---|---|---|---|---|
| | (avg) | InA | InF | InG | InK | InR | InV | InY | InQ | InD | InE | InH | InM | InT | InW |
| IC50 mutein TLN | | 13.887 | 11.627 | 10000 | 5.656 | 3.816 | 11.119 | 10.893 | 10.371 | 10.599 | 43.488 | 5.344 | 4.795 | 10.841 | 10.599 |
| IC50 wt TLN | 12.51 | 12.408 | 12.555 | 12.553 | 12.545 | 12.581 | 12.551 | 12.553 | 12.552 | 12.53 | 12.526 | 12.379 | 12.415 | 12.432 | 12.53 |
| IC50 mutein PLN | | 74.4 | 105.107 | 28.916 | 45.25 | 16.069 | 22.275 | 34.934 | 15.404 | 47.007 | 5000 | 71.648 | 11.097 | 103.341 | 62.954 |
| IC50 wt PLN | 21.91 | 22.009 | 22.338 | 21.56 | 22.028 | 22.261 | 22.087 | 22.167 | 21.925 | 20.857 | 21.56 | 22.032 | 21.961 | 21.923 | 22.076 |
| ratio TLN:PLN | 0.57 | 0.19 | 0.11 | 345.83 | 0.12 | 0.24 | 0.50 | 0.31 | 0.67 | 0.23 | 0.01 | 0.07 | 0.43 | 0.10 | 0.17 |

| | wt | Pos35 | | | | | | |
|---|---|---|---|---|---|---|---|---|
| | (avg) | InA | InF | InL | InV | InM | InW | InY |
| IC50 mutein TLN | | 34.34 | 13.26 | 9.06 | 14.93 | 12.55 | 14.10 | 16.25 |
| IC50 wt TLN | 12.57 | 12.58 | 12.58 | 12.57 | 12.60 | 12.55 | 12.58 | 12.55 |
| IC50 mutein PLN | | 259.77 | 16.27 | 8.26 | 63.55 | 8.22 | 13.92 | 12.43 |
| IC50 wt PLN | 21.98 | 21.81 | 21.95 | 21.91 | 22.19 | 21.94 | 22.12 | 21.95 |
| ratio TLN:PLN | 0.57 | 0.13 | 0.81 | 1.10 | 0.23 | 1.53 | 1.01 | 1.31 |

TABLE 2

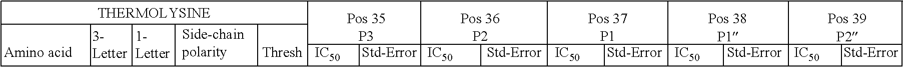

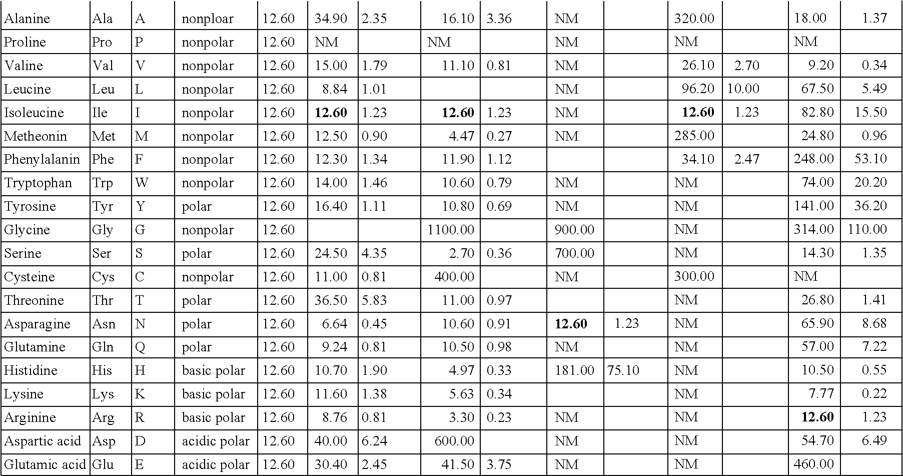

NM = not measurable
Grey: Higher IC₅₀ than wildtype (wildtype in bold and grey)

### EXAMPLE 1

Total RNA Isolation and cDNA Synthesis

Last instar larvae were used for immunization using the following solutions: (4.05*10⁴ cfu/ml) Escherichia coli strain BL21 (DE3) (Invitrogen, Carlsbad, Calif.) and (0.7*10⁴ cfu/ml) Micrococcus luteus (DMSZ Reference number 495). Ten microliters of sample volume from each solution was injected dorsolaterally into the hemocoel using 1-ml disposable syringes and 0.4-mm needles mounted on a microapplicator. Larvae were homogenized at 8 h postinjection for total RNA isolation using the TRI-zol reagent (Invitrogen, Germany) according to manufacturer's instructions. cDNA was synthesized using the OneStep RT-PCR System (Qiagen, Germany) according to the manufacturer's instructions.

### EXAMPLE 2

### Preparation of IMPIs (Expression and Purification)

1. Construction of a Heterologous Expression System
The T7-based expression system, pET-41a(+), which allows protein expression upon induction of the T7-polymerase in Escherichia coli cells by IPTG, was chosen to obtain strong and reproducible expression of the recombinant protein. The sequence of the mature IMPIα was amplified using the forward primer (5′-GA-TAGTCCTAATTTGTAACGGTGGACAC-3′) and the reverse primer (5 CTAC-GAACGTATTTTAGGACAGTCTTTTATCG-3′). The fragment was cloned into the PshAI site of vector pET-41a(+), which was then designated p41IMPIα and transformed into E. coli Rosetta-gami 2 pLysS (Novagen, Germany). The resulting clones were verified via sequencing (Eurofins MWG Operon, Germany)
2. Expression and Production of Recombinant IMPIα
   i. Preparatory Culture: LB-medium containing 1% (w/v) Glucose, Cm₃₄, Kan₅₀ and Tc_{12,5} was inoculated with cell material from a colony and cultivation started on an orbital shaker at 32° C. up to a OD₆₀₀ of 1. The preparatory culture was subsequently stored at 4° C. in a refrigerator.
   ii. Main culture: LB-Medium containing 1% (w/v) Glucose, Cm₃₄, Kan₅₀, was inoculated with 3%(v/v) of the preparatory culture and cultivation started on an orbital shaker at 32° C. up to a OD₆₀₀ of 1. Upon reaching an OD₆₀₀ of 1 the expression of the recombinant protein was induced with 1 mM IPTG. The main culture was subsequently incubated for further 3 hours at 32° C. on an orbital shaker.
   iii. Cells were harvested from liquid culture by centrifugation at 10,000 g for 10 min using a centrifuge tube of known weight. The pellet was decanted and allowed to drain, removing a maximum amount of liquid before the wet weight of the pellet was determined.
   iv. The cell pellet was resuspended in BugBuster® Reagent (Novagen, Germany) at room temperature by pipeting and gentle vortexing. 5 ml reagent per g wet cell paste was used. 1 μl (25 units) Benzonase® Nuclease (Novagen, Germany) per 1 ml of BugBuster® Reagent was added.
   v. The suspension obtained in step (iv) of this protocol was incubated at room temperature on a shaking platform at a slow setting for 20 min. Care was taken that the extract was not viscous at the end of the incubation.
   vi. Insoluble cell debris was removed by centrifugation at 16,000×g for 20 min at 4° C.
   vii. The supernatant was transferred to a fresh tube. The soluble extract was applied directly to GST•Bind™ Resin (Novagen, Germany).
   viii. Affinity chromatography was performed according to manufacturer's instructions (GE Healthcare Life Sciences.
3. Main culture: LB-Medium containing 1% (w/v) Glucose, Cm₃₄, Kan₅₀ was inoculated with 3% (v/v) of the preparatory culture and cultivation started on an orbital shaker at 32° C. until an OD₆₀₀ of 1 was reached. At 1 OD the expression of the recombinant protein was induced with 1 mM IPTG. The main culture was subsequently incubated for further 3 hours at 32° C. on an orbital shaker.

### EXAMPLE 3

### Bioinformatics Analysis

Protein motifs were identified using SMART (European Molecular Biology Laboratory, Heidelberg, Germany) and the Conserved Domain Database from NCBI (National Center for Biotechnology Information, Bethesda, Md., USA). The signal peptide was predicted using SignalP (Center for Biological Sequence Analysis, Technical University of Denmark), and the theoretical isolelectric point and molecular weight were predicted using Compute pI/MW (ExPASy, Swiss Institute of Bioinformatics). Cysteine disulfide bonding state and connectivity prediction was done by using DISULFIND (Dipartimento di Ingegneria dell'Informazione, Universita di Firenze, Firenze, Italy). Sequence similarity was analysed by BLAST from NCBI (National Center for Biotechnology Information, Bethesda, Md., USA). Multi-sequence alignment was generated using Vector NTI 9.0 (Invitrogen, Germany). Homology modeling was performed by the CPHmodels 3.0 server (Center for Biological Sequence Analysis, Technical University of Denmark) using the Swiss-Model server (ExPASy, Swiss Institute of Bioinformatics) for structure assessment. All models were energy minimized by using GROMOS force field (GROMACS, Stockholm, Sweden).

The subsequent table lists the sequences printed in the ensuing sequence protocol. The leading number denotes the SEQ ID NO for the nucleotide sequence, the subsequent even number would denote the SEQ ID NO of the respective peptide sequence.

1 IMPIalpha (wild type or wtlMPIalpha)

3 IMPIbeta (wild type or wt IMPIbeta)

5 GST/IMPIalpha

7 GST/IMPIbeta

9: IMPIalpha Pos37 NnG Pos38 InL Pos39 RnA

11: GST/IMPIalpha Pos37 NnG Pos38 InL Pos39 RnA

13: IMPIalpha Pos52 RnK

15: GST/IMPIalpha Pos52 RnK

17: IMPI Pos35 InL

19: IMPI Pos35 InM

21: IMPI Pos35 InF

23: IMPI Pos35 InC

25: IMPI Pos35 InN

27: IMPI Pos35 InQ

29: IMPI Pos35 InH

31: IMPI Pos35 InK

33: IMPI Pos35 InR

35: IMPI Pos36 InV

37: IMPI Pos36 InM

39: IMPI Pos36 InF

41: IMPI Pos36 InW

43: IMPI Pos36 InY

45: IMPI Pos36 InS

47: IMPI Pos36 InT

49: IMPI Pos36 InN

51: IMPI Pos36 InQ

53: IMPI Pos36 InH

55: IMPI Pos36 InR

57: IMPI Pos36 InK

59: IMPI Pos39 InV

61: IMPI Pos39 InK

63: IMPI Pos35 InW

65: IMPI Pos35 InY

67: IMPI Pos39 RnA

69: IMPI Pos35 InC

71: IMPI Pos35 InK

73: IMPI Pos35 InR

75: IMPI Pos35 InL

77: IMPI Pos35 InM

79: IMPI Pos35 InF

81: IMPI Pos35 InQ

83: IMPI Pos35 InH

85: GST/IMPI Pos35 InN

87: GST/IMPI Pos36 InS

89: GST/IMPI Pos39 InK

91: GST/IMPI Pos35 InC

93: IMPI signal peptide,

95: IMPI like (Solenopsis, Peptide only, no nucleotide sequence provided)

### REFERENCES

Adekoya O, Sylte I. The thermolysin family (M4) of enzymes: therapeutic and bio-technological potential. Chem Biol Drug Des 2009; 73:7-16.

Armstrong P B. Proteases and protease inhibitors: a balance of activities in host-pathogen interaction. Immunobiol 2006; 211:263-81.

Arnold F. H., Georgiou G. (Eds.) “Directed Enzyme Evolution: Screening and Selection Methods (Methods in Molecular Biology) Springer 2003, ISBN-13 978-1588292865

Arnold F. H., Georgiou G. (Eds.) “Directed Evolution Library Creation: Methods and Protocols (Methods in Molecular Biology) Springer 2003, ISBN-13 978-1588292858

Barret A. J., Rawling N. D. Woessner J. F. (Eds.) (2004) Handbook of Proteolytic Enzymes, Academic Press, Inc., ISBN-13 978-0120796106.

Biela A., Nasief N. N., Betz M., Heine A., Hangauer D., Klebe G. “Dissecting the Hydrophobic Effect on the Molecular Level: The Role of Water, Enthalpy, and Entropy in Ligand Binding to Thermolysin” Angew. Chem. Int. Ed. 2013, 52, 1822 -1828 2013 Wiley-VCH Verlag GmbH & Co. KGaA, Weinheim

Cathcart G. R., Greer B., Harriott P., Lynas J. F., Gilmore B. F., Walker B. “Novel Inhibitors of the Pseudomonas aeruginosa Virulence Factor LasB: a Potential Therapeutic Approach for the Attenuation of Virulence Mechanisms in Pseudomonal Infection” Antimicrob Agents Chemother. 2011 June; 55(6):2670-8

Ceroni A., Passerini A., Vullo A., Frasconi P. “DISULFIND: a Disulfide Bonding State and Cysteine Connectivity Prediction Server”, Nucleic Acids Research, 34 (2006) W177--W181

Chen, Ch.-H., Miller, M. A., Sarkar A., Beste M. T., Isaacson K. B., Lauffenburger D. A., Griffith L. G. and Han J. “Multiplexed Protease Activity Assay for Low-Volume Clinical Samples Using Droplet-Based Microfluidics and Its Application to Endometriosis”, JACS 2013, 135 (5), pp 1645-1648, DOI: 10.1021/ja307866z

Chung M., Popova T. G., Millis B. A., Mukherjee D. V., Zhou W., Liotta L. A., Petricoin E. F., Chandhoke V., Bailey Ch., Popov S. G., “Secreted Neutral Metalloproteases of Bacillus anthracis as Candidate Pathogenic Factors” JBC 281, 42, (2006) 31408-31418, DOI 10.1074/jbc.M605526200

Chung M., Popova T. G., Jorgensen S. C., Dong L., Chandhoke V., Bailey Ch. L., Popov S. G., “Degradation of Circulating von Willebrand Factor and Its Regulator ADAMTS13 Implicates Secreted Bacillus anthracis Metalloproteases in Anthrax Consumptive Coagulopathy” JBC 283, 15, (2008). 9531-9542, DOI 10.1074/jbc.M705871200

Chung M., Jorgensen S. C., Tonry J. H., Kashanchi F., Bailey Ch. L., Popov S. G., “Secreted Bacillus anthracis proteases target the host ¢brinolytic system” FEMS Immunol Med Microbiol 62 (2011) 1-9, DOI:10.1111/j.1574-695X.2011.00798.x

Clermont A, Wedde M, Seitz V, Podsiadlowski L, Hummel M, Vilcinskas A. Cloning and expression of an inhibitor against microbial metalloproteinases from insects (IMPI) contributing to innate immunity. Biochem J 382 (2004); 315

Duthie E. S. and Lorenz L. “Protease Inhibitors 2.Bacterial Proteases and their inhibitors” Biochem J. 44(1949) 173-178

Fischer-Durand N., Salmain M., Rudolf B, Dai L., Jugé L., Guérineau V., Laprévote O., Vessières A, Jaouen G. “Site-specific conjugation of metal carbonyl dendrimer to antibody and its use as detection reagent in immunoassay” Analytical Biochemistry 407(2) (2010), 211-219, doi:10.1016/j.ab.2010.08.027

Handbook of Proteolytic Enzymes, 3rd Edn chapter 111, pp 540-553, 2013 Elsevier Ltd. ISBN: 978-0-12-382219-2 DOI: http://dx.doi.org/10.1016/B978-0-12-382219-2.00111-3

Hiraga K, Seeram S S, Tate S, Tanaka N, Kainosho M, Oda K Mutational analysis of the reactive site loop of Streptomyces metalloproteinase inhibitor, SMPI. J Biochem. 1999 Jan;125(1):202-9.

Holden H. M., Tronrud D. E., Monzingo A. R., Weaver L. H. and Matthews B. W. Slow- and Fast-Binding Inhibitors of Thermolysin Display Different Modes of Binding: Crystallographic Analysis of Extended Phosphonamidate Transition-State Analogues Bio-chemistry 26(1987) 8542-8553

Junutula J. R., Raab H., Clark S http://www.nature.com/nbt/journal/v26/n8/full/nbt.1480.html-a1, Bhakta S. Leipold D. D. Weir S., Chen Y, Simpson M., Tsai S. P., Dennis M. S., Lu Y., Meng Y. G., Ng C., Yang J., Lee Ch. C., Duenas E., Gorrell J., Katta V., Kim A., McDorman K., Flagella K., Venook R., Ross S., Spencer S. D., Wong W. L., Lowman H. B., Vandlen R., Sliwkowski M. X., Scheller R. H., Polakis P. Mallet W. “Site specific conjugation of a ytotoxic drug to an antibody improves the therapeutic index” Nature Biotechnology 26 (2008) 925-932 doi:10.1038/nbt.1480

Khan M. T. H., Fuskevaag O. M. and Sylte I. Discovery of Potent Thermolysin Inhibitors Using Structure Based Virtual Screening and Binding Assays. Journal of Medicinal Chemistry 52 (2009) 48-61

Khan M. T. H., Khan R., Wuxiuer Y., Arfan M., Ahmed M. and Sylte I. Identification of novel quinazolin-4(3H)-ones as inhibitors of thermolysin, the prototype of the M4 family of proteinases. Bioorganic & Medicinal Chemistry 18 (2010) 4317-4327

Oda k., Koyama T. and Murao S. Purification and properties of a proteinaceous metallo-proteinase inhibitor from Streptomyces nigrescens TK-23, Biochim. Biophys. Acta 571

(1979), pp. 147-156

Otto M. “Bacterial Sensing of Antimicrobial Peptides”, in Collin M, Schuch R (eds): Bacterial Sensing and Signaling. Contrib Microbiol.16, Basel, Karger, 2009,136-149 DOI: 10.1159/00021937

Panchal R. G., Hermone A. R., Nguyen T. L., Wong T. Y., Schwarzenbacher R., Schmidt J., Lane D., McGrath C., Turk B. E., Burnett J., Aman M. J., Little S., Sausville E. A., Zaharevitz D. W., Cantley L. C., Liddington R. C., Gussio R., Bavari S., “Identification of small molecule inhibitors of anthrax lethal factor” Nature Structural & Molecular Biology 11,67-72 (2004) doi:10.1038/nsmb711

Peinado J. R., Kacprzak M. M., Leppla S. H., Lindberg I., “Cross-inhibition between furin and lethal factor inhibitors”, Biochemical and Biophysical Research Communications (2004)321 (3), 601-605 DOI: 10.1016/j.bbrc.2004.07.012

Popov S. G., Popova T. G., Hopkins S., Weinstein R. S., MacAfee R., Fryxell K. J., Chandhoke V., Bailey Ch., Alibek K. “Effective antiprotease-antibiotic treatment of experimental Anthrax” BMC Infectious Diseases 2005, 5:25 doi:10.1186/1471-2334-5-25

Potempa J, Robert N P. Corruption of innate immunity by bacterial proteases. J Innate Immun 2009; 1:70-87

Roy J., Kumar U. Ch., Machiraju P. K., Muttineni R. K., Kumar S. B. V. S., Gundla R., Dayam R., Jagarlapudi S. A. R. P. “Insilico studies on anthrax lethal factor inhibitors: Pharmacophore modeling and virtual screening approaches towards designing of novel inhibitors for a killer” Journal of Molecular Graphics and Modelling 29, 2, 2010, 256-265 doi:10.1016/j.jmgm.2010.07.002

Schmidtchen A., Holst E., Tapper H., Bjorck, L. Elastase-producing Pseudomonas aeruginosa degrade plasma proteins and extracellular products of human skin and fibroblasts, and inhibit fibroblast growth Microb. Pathog. 34 (2003) 47-55

Smith A. W., Chahal B., French G. L. The human gastric pathogen Helicobacter pylori has a gene encoding an enzyme first classified as a mucinase in Vibrio cholerae Mol. Microbiol. 13 (1994) 153-160

Snell K., Holder I. A., Leppla S. A. and Saelinger C. B. Role of Exotoxin and Protease as Possible Virulence Factors in Experimental Infections with Pseudomonas aeruginosa Infection and Immunity 19 (1978) 839-845

Strohal R. Dissemond J., Hastermann G., Herberger K., Läuchli S., Luch G., Mayer D., Neubert T., Storck M. “The role of a point-of-care protease test in wound diagnostics”, WundManagement 2012, pp 9-11.

Tonello F., Seveso M., Marin O., Mock M., Montecucco C., “Pharmacology: Screening Inhibitors of Anthrax Lethal Factor” Nature 418, 386 (July 2002) doi :10.1038/418386a

Tronrud, D. E., Holden, H. M., and Matthews, B. W. “Structures of Two Thermolysin-Inhibitor Complexes That Differ by a Single Hydrogen Bond” Science 235 (1987)571-574

Tsuru D, Fujita Y, Morikawa S, Ito K, Yoshimoto T. “Degradation of streptomyces metalloprotease inhibitor (SMPI) by neutral protease from Bacillus subtilis var. amylosacchariticus” Biosci Biotechnol Biochem. 1992 Aug;56(8):1275-8.

Turk B. E., Wong T. Y., Schwarzenbacher R.; Jarrell E. T., Leppla S. H., Collier R. J., Liddington R. C., Cantley L. C., “The structural basis for substrate and inhibitor selectivity of the anthrax lethal factor” Nature Structural & Molecular Biology 11, 60-66 (2003) doi:10.1038/nsmb708

Vilcinskas A. Coevolution between pathogen-derived proteinases and proteinase inhibitors of host insects. Virulence 2010; 1(3): 206-14

Wedde M, Weise C, Kopacek P, Franke P, Vilcinskas A. Purification and characterization of an inducible metalloprotease inhibitor from the hemolymph of greater wax moth larvae, Galleria mellonella. Eur J Biochem 1998; 255:534-43

Xiong http://www.sciencedirect.com/science/article/pii/S0960894X05013855-aff1 Y., Wiltsie J., Woods A., Guo J., Pivnichny J. V., Tang W.,

Bansal http://www.sciencedirect.com/science/article/pii/S0960894X05013855-aff1 A., Cummings R. T., Cunningham http//www.sciencedirect.com/science/article/pii/S0960894X0501.385.5-aff1 B. R., Friedlander A. M., Douglas http://www.sciencedirect.com/science/article/pii/S0960894X05013855-aff1 C. M., Salowe S. P., Zaller D. M., Scolnick E. M., Schmatz D. M., Bartizal K., Hermes J. D., MacCoss M., Chapman K. T., “The discovery of a potent and selective lethal factor inhibitor for adjunct therapy of anthrax infection”, Bioorganic & Medicinal Chemistry Letters 16(4) 2006, 964-968, doi:10.1016/j.bmcl.2005.10.088

1. A polypeptide having at least 70% homology, in particular 80%, 90% or 95% homology to the polypeptide of SEQ ID NO: 2 representing the wild-type of the protein insect metalloproteinase inhibitor IMPIα and having at least one mutation at position 35, 36 and/or 39 of the amino acid sequence of IMPIα and the polypeptide having an IC₅₀ value to thermolysine of less than the IC₅₀ value of IMPIα wherein
the nonpolar amino acid isoleucine at position 35 of IMPIα is replaced either by a nonpolar amino acid selected from the group consisting of leucine, methionine and phenylalanine or by polar amino acid selected from the group consisting of cysteine, asparagine, glutamine, histidine, lysine and arginine; and/or
the nonpolar amino acid isoleucine at position 36 of IMPIα is replaced either by a nonpolar amino acid selected from the group consisting of valine, phenylalanine and tryptophan or by polar amino acid selected from the group consisting of tyrosine, serine, threonine, asparagine, glutamine, histidine, lysine and arginine; and/or
the polar amino acid position 39 of IMPIα is replaced either by the nonpolar amino acid valine or by the polar amino acids histidine or lysine.

2. The polypeptide of claim 1 having the amino acid sequences of SEQ ID NOs: 10, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84.

3. The polypeptide of claim 1 having chemical modifications in the side chain or at the N and/or C terminal for improving biological or chemical properties such as bio availability, stability, effectivity or comprising a detectable label.

4. A fusion polypeptide comprising a wtIMPIα, wtIMPIβ and/or a polypeptide of claim 1 fused with at least one polypeptide having a physiological function selected from the group consisting of an antibody or antibody fragment, scaffolds, functional peptides, peptides useful for diagnostic applications, peptide tags enabling immobilization on technical surfaces, or a transferase.

5. The fusion polypeptide of claim 4 being linked by a peptide of 1 to 100 amino acids in length.

6. The fusion polypeptide of claim 4 wherein a chemical linking group conjugates the polypeptide of claim 1 and the at least one polypeptide having a physiological function.

7. A polynucleotide coding for the polypeptide claim 1.

8. The polynucleotide of claim 7 operably linked to the heterologous promoter.

9. A host cell comprising the recombinant nucleic acid of claim 7.

10. A method of producing a polypeptide comprising culturing the host cell of claim 9, expression of said nucleic acid in the host cell and isolation of the polypeptide.

11. A kit for diagnosis comprising a peptide of claim 3.

12. A pharmaceutical composition comprising the
polypeptide having at least 70% homology, in particular 80%, 90% or 95% homology to the polypeptide of SEQ ID NO: 2 representing the wild-type of the protein insect metalloproteinase inhibitor IMPIα, in particular having at least one mutation at position 35, 36 and/or 39 of the amino acid sequence of IMPIα, wherein
the nonpolar amino acid isoleucine at position 35 of IMPIα is replaced either by a nonpolar amino acid selected from the group consisting of leucine, methionine and phenylalanine or by polar amino acid selected from the group consisting of cysteine, asparagine, glutamine, histidine, lysine and arginine; and/or
the nonpolar amino acid isoleucine at position 36 of IMPIα is replaced either by a nonpolar amino acid selected from the group consisting of valine, phenylalanine and tryptophan or by polar amino acid selected from the group consisting of tyrosine, serine, threonine, asparagine, glutamine, histidine, lysine and arginine; and/or
the polar amino acid position 39 of IMPIα is replaced either by the nonpolar amino acid valine or by the polar amino acids histidine or lysine, and/or the fusion polypeptide of claim 4.

13. The polypeptide having at least 70% homology, in particular 80%, 90% or 95% homology to the polypeptide of SEQ ID NO: 2 representing the wild-type of the protein insect metalloproteinase inhibitor IMPIα and having at least one mutation at position 35, 36 and/or 39 of the amino acid sequence of IMPIα, wherein
the nonpolar amino acid isoleucine at position 35 of IMPIα is replaced either by a nonpolar amino acid selected from the group consisting of leucine, methionine and phenylalanine or by polar amino acid selected from the group consisting of cysteine, asparagine, glutamine, histidine, lysine and arginine; and/or
the nonpolar amino acid isoleucine at position 36 of IMPIα is replaced either by a nonpolar amino acid selected from the group consisting of valine, phenylalanine and tryptophan or by polar amino acid selected from the group consisting of tyrosine, serine, threonine, asparagine, glutamine, histidine, lysine and arginine; and/or
the polar amino acid position 39 of IMPIα is replaced either by the nonpolar amino acid valine or by the polar amino acids histidine or lysine, or the fusion polypeptide of claim 4 for use in the treatment of an animal or human infected by microorganisms secreting bacterial toxins of the M4 or Metzincin family of metalloproteinases.

14. A method for detecting the presence or activity of proteases belonging to the M4 family in a sample using the polypeptide of claim 3.

15. The fusion polypeptide of claim 4 wherein the scaffold is selected from the group consisting of lipocalin, ankyrin, fibronectin, transferrin, tetranectin, adnectin, albumin, uteroglobin, and protein A.

16. A polynucleotide coding for the fusion polypeptide of claim 5.

17. The pharmaceutical composition of claim 12 wherein the polar amino acid position 39 of IMPIα is replaced by a polynucleotide comprising a section coding for the polypeptide of claim 1.

18. The polypeptide of claim 13 wherein the polar amino acid position 39 of IMPIα is replaced by the polynucleotide of claim 7.

19. The polypeptide of claim 13 wherein the bacterial toxins of the M4 or Metzincin family of metalloproteinases are selected from the group thermolysine, aureolysin, bacillolysin, pseudolysin, vibriolysin or anthrax npr599.

20. A method for detection of the presence or activity of proteases belonging to the M4 family in a sample using a fusion polypeptide of claim 4.
